(19) United States
(12) Patent Application Publication
(10) Pub. No.: US 2024/0124828 A1
(43) Pub. Date: Apr. 18, 2024

(54) METHOD AND SYSTEM FOR DETERMINING MIGRATION CAPACITY OF CELLS

(71) Applicant: SHANGHAI RUIYU BIOTECH CO., LTD., Shanghai (CN)

(72) Inventors: Yanfang CHEN, Shanghai (CN); Haohan XIA, Shanghai (CN)

(73) Assignee: SHANGHAI RUIYU BIOTECH CO., LTD., Shanghai (CN)

(21) Appl. No.: 18/540,799

(22) Filed: Dec. 14, 2023

Related U.S. Application Data
(63) Continuation of application No. PCT/CN2022/098641, filed on Jun. 14, 2022.

(30) Foreign Application Priority Data
Jun. 17, 2021 (CN) 202110671398.1

(51) Int. Cl. C12M 1/34; C12M 1/36

(52) U.S. Cl. CPC C12M 41/46; C12M 41/40; C12M 41/48

(57) ABSTRACT

The present disclosure relates to a method and a system for determining a cell migration capacity. The method includes performing a scratching operation on cells by a cell scratching device to form at least one scratch and photographing and analyzing the at least one scratch by a cell image analysis device to determine a migration capacity of the cells.

### CROSS REFERENCE TO RELATED APPLICATIONS

This application is a continuation of International Application No. PCT/CN2022/098641, filed on Jun. 14, 2022, which claims priority to Chinese application No. 202110671398.1, filed Jun. 17, 2021, and the entire contents of each of which are incorporated herein by reference.

### TECHNICAL FIELD

The present disclosure relates to the field of biotechnology, and in particular, to a method and system for determining migration capacity of cells.

### BACKGROUND

Cell migration is closely related to the occurrence and development of cancer, and cell migration capacity is one of the main indicators to measure a metastasis ability of cancer cells. The study on the cell migration capacity, on the one hand, can deepen the understanding of cell migration behavior, and on the other hand, it is of great value for the therapeutic exploration of diseases that are closely related to cell migration such as cancer. The cell scratching experiment is one of the commonly used manners to study the cell migration capacity. The producing of a scratch, the acquisition of images during the cell migration, and the processing and analyzing of image data are key steps of the cell scratching experiment. Therefore, it is desirable to provide a method and a system for determining migration capacity of cells, which can ensure a homogeneous stability of the scratch and a reproducibility of the cell scratching experiment, so that the migration capacity of cells can be detected efficiently, comprehensively, and accurately.

### SUMMARY

In a first aspect of the present disclosure, a method for determining a cell migration capacity is provided. The method may include performing a scratching operation on cells through a cell scratching device to form at least one scratch and photographing and analyzing the at least one scratch by a cell image analysis device to determine a migration capacity of the cells.

In some embodiments, the photographing and analyzing the at least one scratch by a cell image analysis device to determine the migration capacity of the cell includes positioning and photographing the at least one scratch by the cell image analysis device to obtain at least one image of the at least one scratch; and determining the migration capacity of the cells based on the at least one image of the at least one scratch.

In some embodiments, the cell scratching device includes a cultivation portion and a scratching portion. The cultivation portion may be configured to cultivate the cells. The scratching portion may include a scratching cover plate and a scratching member. The scratching cover plate may include a base plate, a connecting member, and a position-limiting structure that are connected sequentially. The base plate and the position-limiting structure may be respectively located at two ends of the connecting member, and the base plate may be provided with at least one scratching gap.

In some embodiments, the performing a scratching operation on the cells through a cell scratching device to form at least one scratch includes securing the scratching cover plate to the cultivation portion through the position-limiting structure; and performing the scratching operation on the cells by inserting the scratching member into the at least one scratching gap and moving the scratching member along one end of the at least one scratching gap to the other end of the at least one scratching gap. A position of the at least one scratch may be determined by a position of the at least one scratching gap.

In some embodiments, the photographing and analyzing the at least one scratch by a cell image analysis device to determine the migration capacity of the cells includes automatically photographing, through the cell image analysis device, the at least one scratch based on the photographing parameter and the position of the at least one scratch to obtain a plurality of images of the at least one scratch, wherein each image of the plurality of images corresponds to a preset time point; and determining the migration capacity of the cells based on the plurality of images of the at least one scratch and a plurality of preset time points corresponding to the plurality of images.

In some embodiments, a plurality of scratches is formed after the scratching operation is performed on the cells. The plurality of scratches may include a first scratch and a second scratch. The automatically photographing, through the cell image analysis device, the at least one scratch based on the photographing parameter and the position of the at least one scratch may include controlling the cell image analysis device to automatically photograph the first scratch based on the photographing parameter and a position of the first scratch; determining a positional relationship between the first scratch and the second scratch based on a positional relationship between a first scratching gap corresponding to the first scratch and a second scratching gap corresponding to the second scratch; and controlling the cell image analysis device to automatically photograph the second scratch based on the positional relationship between the first scratch and the second scratch and the photographing parameter.

In some embodiments, the cell image analysis device includes a sample stage and a photographing module. The automatically photographing, through the cell image analysis device, the at least one scratch based on the photographing parameter and the position of the at least one scratch to obtain a plurality of images of the at least one scratch may include at each of the plurality of preset time points, positioning the cultivation portion of the cell scratching device at a target position of the sample stage; and controlling the photographing module to automatically photograph the at least one scratch based on the photographing parameter and the position of the at least one scratch to obtain at least one image of the at least one scratch at the preset time point.

In some embodiments, wherein the controlling the photographing module to automatically photograph the at least one scratch based on the photographing parameter and the position of the at least one scratch to obtain the at least one image of the at least one scratch at the preset time point includes controlling the sample stage or the photographing module to move along a direction parallel to an extending direction of the at least one scratch to obtain a plurality of images of multiple view fields of the at least one scratch along the extending direction.

In some embodiments, wherein a plurality of scratches is formed after the scratching operation is performed on the cells. The plurality of scratches may extend in a direction parallel to each other. The controlling the photographing module to automatically photograph the at least one scratch based on the photographing parameter and the position of the at least one scratch to obtain at least one image of the at least one scratch at the preset time point includes controlling the sample stage or the photographing module to move along a direction perpendicular to an extending direction of the plurality of scratches to obtain a plurality of images of the plurality of scratches.

In some embodiments, wherein the determining the migration capacity of the cells based on the plurality of images of the at least one scratch and a plurality of preset time points corresponding to the plurality of images includes determining a migration distance or a migration area of the cells based on the plurality of images; and determining the migration capacity of the cells based on the migration distance or the migration area, and the plurality of preset time points.

In a second aspect of the present disclosure, a system for determining a cell migration capacity is provided. The system may include a cell scratching device and a cell image analysis device. The cell scratching device may be configured to perform a scratching operation on cells to form at least one scratch. The cell image analysis device may be configured to photograph and analyze the at least one scratch to determine the migration capacity of the cells.

In some embodiments, the cell image analysis device includes a photographing module configured to position and photograph the at least one scratch to obtain at least one image of the at least one scratch.

In some embodiments, the cell image analysis device includes an analyzing module configured to determine the migration capacity of the cells based on the at least one image of the at least one scratch.

In some embodiments, the cell image analysis device includes a sample stage configured to position a cultivation portion of the cell scratching device.

In some embodiments, the cell image analysis device includes a controlling module configured to control a sample stage and/or a photographing module.

In some embodiments, the cell scratching device includes a scratching portion configured to perform the scratching operation on the cells. The scratching portion may include a scratching cover plate. The scratching cover plate may include a base plate, a connecting member, and a position-limiting structure that are connected sequentially. The base plate and the position-limiting structure may be respectively located at two ends of the connecting member. The base plate may be provided with a plurality of scratching gaps. The plurality of scratching gaps may be parallel to each other and uniformly distributed at intervals. The scratching cover plate may be installed and fixed by the position-limiting structure during the scratching operation.

In a third aspect of the present disclosure, a control system for determining a cell migration capacity is provided. The control system may include at least one storage device configured to store a set of instructions and at least one processor being in communication with the at least one storage device. When executing the stored instructions, the at least one processor causes the system to execute the method for determining the cell migration capacity provided in the first aspect of the present disclosure.

In a fourth aspect of the present disclosure, use of the method provided in the first aspect of the present disclosure, the system provided in the second aspect of the present disclosure, and the control system provided in the third aspect of the present disclosure is provided.

Additional features may be set forth in part in the description which follows, and in part may become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary application scenario of a system for determining a cell migration capacity according to some embodiments of the present disclosure;

FIG. 2A is a flowchart illustrating an exemplary process for determining a cell migration capacity according to some embodiments of the present disclosure;

FIG. 2B is a flowchart illustrating an exemplary process for determining a cell migration capacity according to some embodiments of the present disclosure;

FIG. 3 is a flowchart illustrating an exemplary process for obtaining a scratching image according to some embodiments of the present disclosure;

FIG. 4A is a schematic diagram illustrating photographing along a direction parallel to an extending direction of a scratch according to some embodiments of the present disclosure;

FIG. 4B is a schematic diagram illustrating photographing along a direction perpendicular to an extending direction of a scratch according to some embodiments of the present disclosure;

FIG. 5 is a flowchart illustrating an exemplary process for determining a cell migration capacity according to some embodiments of the present disclosure;

FIG. 6 is a schematic diagram illustrating an exemplary scratching image according to some embodiments of the present disclosure;

FIG. 7 is a block diagram illustrating an exemplary cell image analysis device according to some embodiments of the present disclosure;

FIG. 8 is a schematic diagram illustrating an exemplary cross-section of a cell scratching device according to some embodiments of the present disclosure;

FIG. 9 is a schematic top view of an exemplary scratching cover plate 120 according to some embodiments of the present disclosure;

FIG. 10 is a schematic top view of an exemplary cultivation portion 110 according to some embodiments of the present disclosure;

FIG. 11 is a schematic diagram illustrating an exemplary structure of the scratching cover plate 120 according to some embodiments of the present disclosure;

FIG. 12 is a schematic diagram illustrating an exemplary matching structure of a scratching member 130 and a scratching gap 1213-1 according to some embodiments of the present disclosure;

FIG. 13 is a schematic diagram illustrating another exemplary matching structure of the scratching member 130 and the scratching gap 1213-1 according to some embodiments of the present disclosure;

FIG. 14 is a schematic diagram illustrating an exemplary structure of a base plate 1213 according to some embodiments of the present disclosure;

FIG. 15 is a schematic diagram illustrating an exemplary structure of the scratching member 130 according to some embodiments of the present disclosure; and

FIG. 16A and FIG. 16B are schematic diagrams illustrating an exemplary connecting structure of a driving member 140 and the scratching member 130 according to some embodiments of the present disclosure.

### DETAILED DESCRIPTION

In order to more clearly illustrate the technical solutions of the embodiments of the present disclosure, the accompanying drawings required to be used in the description of the embodiments will be briefly described below. However, it should be appreciated by those skilled in the art that the present disclosure may be implemented without these details. In other instances, well-known methods, processes, systems, components, and/or circuits have been described at a high level in order to avoid unnecessarily rendering aspects of the present disclosure obscure. It is apparent to those of ordinary skill in the art that various changes can be made to the disclosed embodiments, and without departing from the principles and scope of the present disclosure, the general principles defined herein can be applied to other embodiments and application scenarios. Thus, the present disclosure is not limited to the embodiments shown, but conforms to the broadest possible scope consistent with the scope of the patent application.

The terminology used in the present disclosure is for the purpose of describing particular example embodiments only and is not limiting. As used in the present disclosure, the singular forms “one,” “a,” and “the” may likewise include the plural form, unless the context clearly suggests an exception. It should also be understood that, as used in the present disclosure, the terms “comprising” and “including” only suggest the presence of the features, integers, steps, operations, components, and/or parts, but do not preclude the presence or addition of other features, integers, steps, operations, components, parts, and/or combinations thereof as described above.

It will be appreciated that the present disclosure uses the terms “system,” “engine,” “unit,” “module,” and/or “block” as used herein is a method of distinguishing different components, elements, parts, sections, or assemblies at different levels of hierarchy in ascending order. However, these terms may be replaced by other expressions if the same purpose can be achieved.

These and other features, characteristics, and methods of functioning and operation of the relevant structural elements, as well as combinations of parts and economies of manufacture of the present disclosure may be rendered more apparent in the light of the following description of the accompanying drawings, which all form part of the present disclosure. It should be understood, however, that the accompanying drawings are for illustrative and descriptive purposes only, and are not intended to limit the scope of the present disclosure. It should be understood that the accompanying drawings are not to scale.

The flowcharts used in the present disclosure may illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

Cell migration is one of the basic functions of normal cells, a physiological process of normal growth, and development of an organism, and a movement form prevalent among living cells. Embryonic development, angiogenesis, wound healing, immune response, inflammatory response, atherosclerosis, cancer metastasis and other processes all involve the cell migration.

A cell scratching experiment is the most commonly used and simplest method to analyze a cell migration capacity in the laboratory. When cells grow and fuse into a monolayer, a blank region is artificially created on the fused monolayer, which is called a “scratch.” Cells at an edge of the scratch will gradually move into the blank region to heal the scratch.

Traditional cell scratching experiments generally require that one or more uniform horizontal lines be drawn on a back side (a side that does not contact with the cells) of a plate of a petri dish by means of a marker pen first, and then the cells are cultivated. When a cell layer has been obtained, in a direction of the horizontal lines on the back side of the plate, one or more scratches were made on the cell layer by means of a gun tip or a toothpick. Then, the petri dish was taken out at an appropriate time point, such as after 0, 6, 12, 24 hours, and the scratches were found under a microscope by a marker on the back side of the plate, and a width of the scratches was observed and measured and photographed. Finally, after opening pictures using an image analysis software (e.g., an Image J software), six to eight horizontal lines were randomly selected to calculate an average value of a distance between the cells to obtain the cell migration capacity (e.g., a cell migration rate).

However, traditional cell scratching manners often result in uneven scratches due to uneven force. Additionally, when the scratches are photographed and analyzed, it is difficult to achieve continuous observation of a fixed point with only a marker of a marker pen, which is greatly affected by subjective factors. Photographing under the microscope is time-consuming and cumbersome, and image analysis of the Image J software is less efficient.

One aspect of the present disclosure provides a method for determining a cell migration capacity. The method includes performing a scratching operation on cells using a cell scratching device to form at least one scratch. The method includes automatically photographing, through a cell image analysis device, the at least one scratch based on a preset photographing parameter and a position of the at least one scratch, so as to obtain a plurality of images of the at least one scratch. Each image of the plurality of images corresponds to a preset time point. The preset photographing parameter includes at least one of a sample feed coordinate, a total photographing length of the at least one scratch, or a count of view fields of the at least one scratch. The method further includes determining the migration capacity of the cells based on the plurality of images of the at least one scratch and a plurality of preset time points corresponding to the plurality of images.

Another aspect of the present disclosure provides a system for determining a cell migration capacity. The system includes a cell scratching device and a cell image analysis device. The cell scratching device includes a cultivation portion for cultivating cells and a scratching portion for performing a scratching operation on the cells to form at least one scratch. The cell image analysis device is used for automatically photographing and analyzing the at least one scratch based on a preset photographing parameter and a position of the at least one scratch, so as to determine the migration capacity of the cells.

By using the cell scratching device provided herein to perform the scratching operation on the cells, the homogeneous stability of the scratch and the reproducibility of the scratching experiment can be ensured. By combining the use of the cell scratching device and the cell image analysis device, when analyzing a migration situation of the cells of the scratch, it is possible to accurately locate the scratch without the need for marking with a marker pen. That is, it is possible to take pictures continuously at a fixed point and analyze the pictures to obtain a migration distance and a migration area of the cells, and cumulative observation and analysis of a single scratch and comparative observation and analysis of a plurality of scratches can be realized. Thus, the method for determining the cell migration capacity provided in the present disclosure does not require marking with the marker pen, which overcomes errors due to uneven scratches and manual manipulation of a traditional scratching manner, and can detect the migration capacity of the cells more efficiently, comprehensively, and accurately.

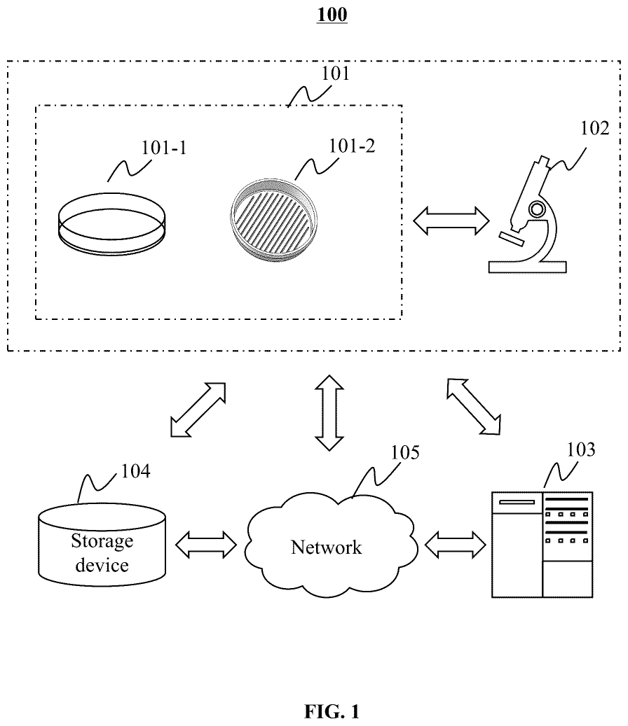

FIG. 1 is a schematic diagram illustrating an exemplary application scenario of a system for determining a cell migration capacity according to some embodiments of the present disclosure. A system 100 for determining the cell migration capacity (also referred to as a control system 100) (also referred to as a system 100 for short) may include a cell scratching device 101, a cell image analysis device 102, a network 105, a storage device 104, and a processing device 103. Components in the system 100 may be inter-connected in various ways. Merely by way of example, the cell scratching device 101 and/or the cell image analysis device 102 may be connected to the processing device 103 or the storage device 104 either directly or via the network 105. As another example, the cell scratching device 101 and the cell image analysis device 102 may be connected to each other directly or via the network 105.

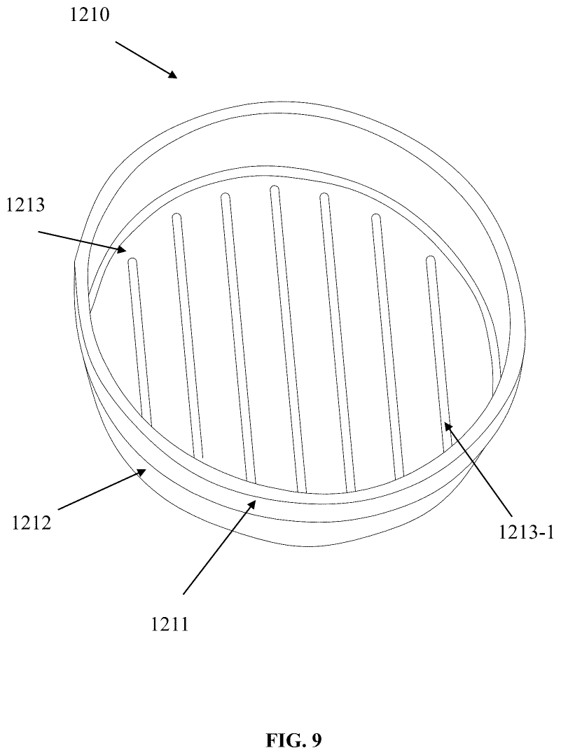

The cell scratching device 101 may be configured to perform a scratching operation on cultivated cells. In some embodiments, the cell scratching device 101 may include a cultivation portion 101-1 and a scratching portion 101-2. The cultivation portion 101-1 (also referred to as a cell cultivation device) may be configured for cell cultivation, and the scratching portion 101-2 may be configured for scratching the cells. In some embodiments, the cultivation portion 101-1 may include a petri dish or a cultivation plate. The petri dish may be made of glass or plastic. In some embodiments, the cultivation portion 101-1 may include a D90 petri dish, a six-well cultivation plate, or the like. In some embodiments, the scratching portion 101-2 may include a scratching cover plate and a scratching member. In some embodiments, the scratching cover plate may include a base plate, a connecting member, and a position-limiting structure that are sequentially connected. The base plate and the position-limiting structure are located at two ends of the connecting member, respectively. More descriptions of the cell scratching device 101 may be found elsewhere in the present disclosure (e.g., FIGS. 8 to 16B and the descriptions thereof).

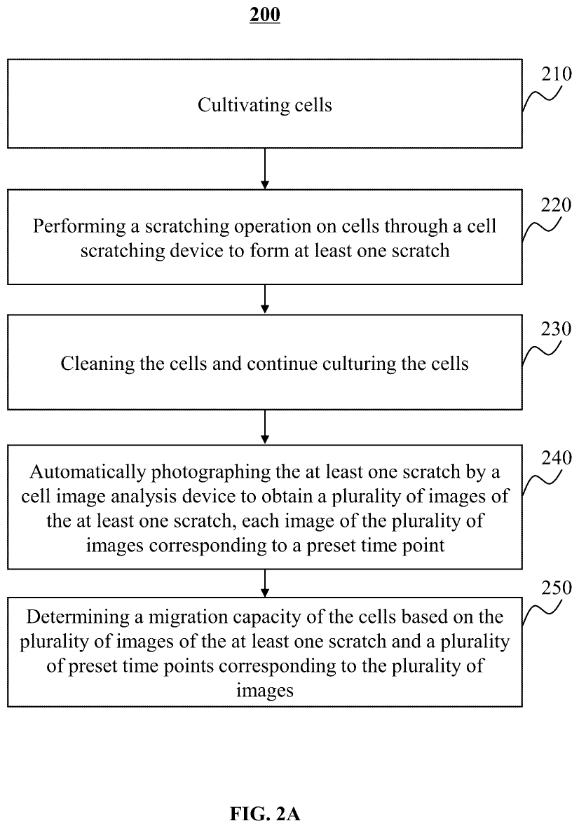

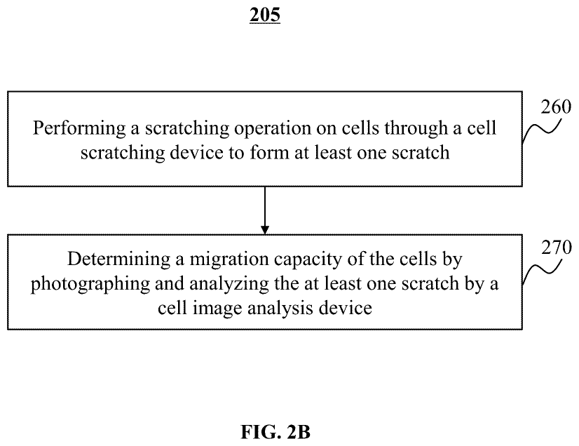

The cell image analysis device 102 may be configured to analyze a physiological activity of the cells by photographing images. For example, the cell image analysis device 102 may be configured to position a scratch, control a photographing trajectory, obtain an image of the scratch, and analyze the image of the scratch to determine the migration capacity of the cells, or the like. In some embodiments, the cell image analysis device 102 may include a sample stage, a photographing module, an analyzing module, a controlling module, or the like, or any combination thereof. More description of the cell image analysis device 102 may be found elsewhere in the present disclosure (e.g., FIGS. 2A to 7 and the descriptions thereof).

The processing device 103 may process data and/or information obtained from other devices or the components of the system 100. The processing device 103 may execute program instructions based on such data, information, and/or processing results to perform one or more of the functions described herein. For example, the processing device 103 may obtain a position of a scratching gap from the cell scratching device 101. As another example, the processing device 103 may obtain the image of the scratch from the cell image analysis device 102. As yet another example, the processing device 103 may determine the migration capacity of the cells based on the image of the scratch. As still yet another example, the processing device 103 may obtain an image analysis result (e.g., the migration capacity of the cells) directly from the cell image analysis device 102. In some embodiments, the processing device 103 may be a single server or a group of servers. The group of servers may be centralized or distributed. In some embodiments, the processing device 103 may be a local component or a remote component relative to one or more other components of the system 100. For example, the processing device 103 may access information and/or data stored in the cell scratching device 101, the cell image analysis device 102, and/or the storage device 104 via the network 105. As another example, the processing device 103 may be directly connected to the cell scratching device 101, the cell image analysis device 102, and/or the storage device 104 to access stored information and/or data. In some embodiments, the processing device 103 may be implemented on a cloud platform. Merely by way of example, the cloud platform may include a private cloud, a public cloud, a hybrid cloud, a community cloud, a distributed cloud, an on-premises cloud, a multi-tier cloud, etc., or any combination thereof. In some embodiments, the processor 103 may include a central processing unit (CPU), an application-specific integrated circuit (ASIC), an application-specific instruction processor (ASIP), a graphics processor (GPU), a physical Processor (PPU), a Digital Signal Processor (DSP), Field Programmable Gate Array (FPGA), Programmable Logic Circuit (PLD), controller, microcontroller unit, Reduced Instruction Set Computer (RISC), microprocessor, etc., or any combination thereof.

The network 105 may connect the components of the system 100 and/or connect the system 100 to an external resource section. The network 105 enables communication between the components of the system 100, and the components of the system 100 and other portions outside of the system 100, to facilitate the exchange of data and/or information. In some embodiments, the network 105 may be any one or more of a wired network or a wireless network. For example, the network 105 may include a cable network, a fiber optic network, a telecommunications network, the Internet, a local area network (LAN), a wide area network (WAN), a wireless local area network (WLAN), a metropolitan area network (MAN), a public switched telephone network (PSTN), a Bluetooth network, a ZigBee network (ZigBee), a near field communication (NFC), an in-device bus, an in-device line, a cable connection, etc., or any combination thereof. The network connection between the various parts may be made in one of the above ways or in multiple ways. In some embodiments, the network may be a variety of topologies, such as point-to-point, shared, centralized, etc., or a combination of topologies.

The storage device 104 may be configured to store data and/or instructions. The storage device 104 may include one or more storage components, each of which may be a stand-alone device or may be part of other devices. In some embodiments, the storage device 104 may include random access memory (RAM), read-only memory (ROM), mass storage, removable memory, volatile read/write memory, or the like, or any combinations thereof. Exemplarily, the mass storage may include disks, optical disks, solid state disks, or the like. In some embodiments, the storage device 104 may be implemented on the cloud platform. Merely by way of example, the cloud platform may include a private cloud, a public cloud, a hybrid cloud, a community cloud, a distributed cloud, an on-premises cloud, a multi-tiered cloud, etc., or any combination thereof.

It should be noted that the above description is provided for illustrative purposes only and is not intended to limit the scope of the present disclosure. For those having ordinary skills in the art, various changes and modifications may be made under the guidance of the contents of the present disclosure. The features, structures, methods, and other characteristics of the exemplary embodiments described in the present disclosure may be combined in various ways to obtain additional and/or alternative exemplary embodiments. However, these changes and modifications do not depart from the scope of the present disclosure. In some embodiments, the cell scratching device 101 and the cell image analysis device 102 may be integrated into a single scratch analyzing device. During performing the scratching operation on the cells, the scratching member (e.g., a scratching needle) is mounted at a fixed position on the sample stage of the scratching analyzing device, and after the cultivation portion is mounted and positioned on the sample stage, the processing device 103 may obtain a position coordinate of the cultivation portion on the sample stage and an installation position coordinate of the scratching member, and the processing process 103 may calculate, based on a scratching scheme, an initial scratching position and an end scratching position of each scratch, and control the cultivation portion on the sample stage to move according to the initial scratching position and the end scratching position to perform the scratching operation by the scratching member. When photographing, it is only necessary to replace the scratching member with a photographing device (e.g., a camera), and take a same movement approach as that of the scratching member to realize the fixed-point photographing of each scratch.

In some embodiments, the storage device 104 and/or the processing device 103 may be integrated into the cell scratching device 101 and/or the cell image analysis device 102. For example, there are corresponding processing devices in each of the cell scratching device 101 and the cell image analysis device 102.

In some embodiments, one or more components of the system 100 may be omitted. For example, the network 105, the storage device 104, and/or the processing device 103 may be omitted. A user may perform the scratching operation using the cell scratching device 101 and input the position of the scratch to the cell image analysis device 102, and the cell image analysis device 102 may photograph and analyze the scratch based on the position of the scratch.

In some embodiments, the system 100 may include one or more other components. For example, the system 100 may include a terminal device (not shown in FIG. 1). The terminal device may include a mobile device, a tablet computer, a laptop computer, etc., or any combination thereof. In some embodiments, the user may use the terminal device to control one or more components (e.g., the cell scratching device 101, the cell image analysis device 102) of the system 100. For example, the user may input, via the terminal device, commands to control the cell scratching device 101 to perform the scratching operation and/or commands to control the cell image analysis device 102 to perform the photographing and analyzing. The cell scratching device 101 may perform the scratching operation based on the commands input by the user. The cell image analysis device 102 may photograph and analyze the scratch based on the commands input by the user. In some embodiments, the terminal device may display data from the system 100. For example, the terminal device may display information such as the position, shape, contour, etc. of the scratch. As another example, the terminal device may display the image of the scratch. As yet another example, the terminal device may display information such as the migration distance, the migration area, the migration capacity, or the like of the cells.

FIG. 2A is a flowchart illustrating an exemplary process for determining a cell migration capacity according to some embodiments of the present disclosure. In some embodiments, at least a portion of a process 200 may be performed by the cell scratching device 101, the cell image analysis device 102, or the processing device 103. For example, the process 200 may be stored as instructions (e.g., an application program) in a storage device (e.g., the storage device 104) and invoked and executed by the cell scratching device 101, the cell image analysis device 102, or the processing device 103. The operation of the process shown below is for illustrative purposes only. In some embodiments, the process 200 may be accomplished utilizing one or more additional operations not described herein and/or without one or more of the operations described herein. Additionally, the order of the operations of the process 200 illustrated in FIG. 2A and described below is not intended to be limiting.

In step 210, a cell is cultivated in a cell cultivation device (e.g., the cultivation portion 101-1).

In some embodiments, cells that grow well in a logarithmic phase are taken and inoculated into the cultivation portion, and the cells are cultivated using a cultivation medium. An amount of the inoculation shall be subject to the fusion rate reaching 100% after 24 hours of cultivation. The fusion rate refers to a percentage of adherent growing cells cultured in a monolayer to the area of the growing region of the cells. When the fusion rate reaches 100%, it means that the cells cover the bottom of the cultivation portion.

In some embodiments, the cultivation medium is removed from the cultivation portion after the cells have reached 100% fusion. For example, a user (e.g., an experimenter) may use a pipette to aspirate the cultivation medium from the cultivation portion, and use a scratching portion (e.g., the scratching portion 101-2) to perform a scratching operation on the cells.

In step 220, the scratching operation is performed on the cells through a cell scratching device (e.g., the scratching portion 101-2) to form at least one scratch.

In some embodiments, the cell scratching device may be a manual scratching device, a semi-automatic scratching device, or a fully automatic scratching device. For example, the scratching portion of the cell scratching device may include a scratching cover plate and a scratching member. The scratching cover plate includes a base plate, a connecting member, and a position-limiting structure that are connected in sequence. The base plate and the position-limiting structure are respectively located at two ends of the connecting member. The base plate is provided with at least one scratching gap. When the user performs the scratching operation using the cell scratching device, the user may fix the cultivation portion in which the cultivation medium is removed on an operation table, and then fix the scratching cover plate on the cultivation portion by the position-limiting structure, and finally, by inserting the scratching member into at least one scratching gap and moving the scratching member along one end of the at least one scratching gap to the other end of the at least one scratching gap to perform the scratching operation on the cells. Specifically, the scratching member may be moved to an end of the scratching gap and inserted into the scratching gap, the scratching member may be slid along the scratching gap to the other end of the scratching gap, and then the scratching member may be taken out (in particular, the scratching member may slide repeatedly to scrape away as many cells from the scratching path as possible). A position of the at least one scratch is determined by a position of the at least one scratching gap. More description of the cell scratching device and the scratching operation using the cell scratching device may be found in FIGS. 8 to 16B and the descriptions thereof.

In step 230, the cells are cleaned and continued to be cultivated in the cell cultivation device (e.g., the cultivation portion 101-1).

After the scratching operation is completed, the user may rinse a surface of the cells several times with a buffer solution (e.g., sterile phosphate buffered saline solution (PBS)) to clean away non-adherent cells produced during the scratching and to make a boundary between the scratch and the cell clear and clean. After the cleaning is completed, a new cultivation medium is added and the cultivation continues. For example, the cells may be placed in a 5% CO₂ incubator at 37° C. to continue the cultivation.

In step 240, automatically photographing the at least one scratch by the cell image analysis device (e.g., the cell image analysis device 102) to obtain a plurality of images of the at least one scratch. In some embodiments, the at least one scratch may be automatically photographed through the cell image analysis device based on a preset photographing parameter and a position of the at least one scratch, so as to obtain the plurality of images of the at least one scratch.

In some embodiments, each image of the plurality of images corresponds to a preset time point. The preset time point may be a time point determined in advance for photographing. For example, the preset time point may be 0 hours, 6 hours, 12 hours, 24 hours, etc., after the scratching operation is performed on the cells.

The preset photographing parameter refers to a relevant parameter used for photographing an image. The preset photographing parameter may be a parameter preset by the user according to a model of the cell cultivation device, a model of the cell image analysis device, a type of the cells, the position of the scratch, an area of the scratch, the precision requirement of the experiment, etc. In some embodiments, the preset photographing parameter may include a sample feed coordinate, a total photographing length of the at least one scratch, a count of view fields of the at least one scratch, etc., or any combination thereof.

The sample feed coordinate refers to a relative position of a zero position of the sample stage and a first photographing view field. In some embodiments, the zero position of the sample stage refers to an initial position of the sample stage. For example, the sample stage may be in the zero position of the sample stage when the cell image analysis device is in an initially unused state. When the cell image analysis device finishes the photographing, the sample stage may return to the zero position. The photographing view field refers to a location on the scratch to be photographed. The first photographing view field refers to a first location on the scratch to be photographed.

In some embodiments, a position coordinate of the first photographing view field of the scratch may be input into the cell image analysis device, and the cell image analysis device may determine the sample feed coordinate of the sample stage based on a zero position coordinate of the sample stage and the position coordinate of the first photographing view field. The cell image analysis device may, based on the sample feed coordinate of the sample stage, control a movement direction and a movement distance of the sample stage, so that a photographing module (e.g., a photographing module 720) of the cell image analysis device may photograph the first photographing view field of the scratch in the cultivation portion on the sample stage.

The total photographing length of the scratch refers to a length of the scratch that needs to be photographed. The count of view fields of the scratch refers to a count of times the scratch is photographed. For example, assuming that a scratch has a length of 5 cm, and setting the total photographing length of the scratch to be 3 cm and the count of view fields to be 10, then it may be determined that starting from a starting position at one end of the scratch, every 0.3 cm is a photographing view field.

In some embodiments, the total photographing length and the count of view fields may be determined based on a photographing situation or a precision requirement of the experiment. For example, in order to improve the precision of the experiment, more experimental data may be obtained by increasing the total photographing length and the count of view fields. As another example, since the uneven tiled cells cause the gap in a scratch to be too small or too large, the scratch does not need to be photographed.

In some embodiments, the user may set the total photographing length and the count of view fields of a first scratch, and the cell image analysis device may automatically generate the total photographing lengths and the counts of view fields of other scratches based on a positional relationship between the other scratches and the first scratch. For example, assuming that a length of the first scratch is 5 cm, the total photographing length of the first scratch is 3 cm, the count of view fields of the first scratch is 10, and a length of a second scratch is equal to the length of the first scratch, the cell image analysis device may automatically set the total photographing length of the second scratch to be 3 cm and the count of view fields of the second scratch to be 10. Assuming that the length of the second scratch is 2.5 cm, the cell image analysis device may automatically set the total photographing length of the second scratch to be 1.5 cm and the count of view fields of the second scratch to be 5.

In some embodiments, the photographing parameter may also include an exposure degree and a photographing focal length, or the like. In some embodiments, the user may predetermine the exposure degree and the photographing focal length based on experience. In some embodiments, the photographing focal length may be dynamically adjusted based on an image photographed in real time. For example, a photographing focal length for optimal imaging may be determined based on the clarity of scratches in a plurality of images photographed in real time. Specifically, a photographing focal length corresponding to an image with the best clarity is determined as the photographing focal length for optimal imaging, and the photographing focal length for optimal imaging is used for photographing other view fields. As another example, due to the manufacturing process of the cultivation portion, it is not possible to ensure that different parts of a bottom of the cultivation portion are at a same distance from a camera. Therefore, it is possible to automatically adjust the focus before photographing each view field, which ensures a better image quality for each photographing. In some embodiments, photographing focal lengths of photographing view fields at other preset time points may be determined based on photographing focal lengths of a plurality of photographing view fields determined at a first preset time point. For example, a plurality of view fields of a scratch is photographed at a preset time point of 0 hours, and a focus of each view field is adjusted and a photographing focal length for optimal imaging is recorded, and when photographing view fields at other preset time points (e.g., 6 hours, 12 hours, 24 hours), the recorded photographing focal length may be obtained and used.

In some embodiments, in order to ensure a consistent image resolution, images of all scratches may be obtained using a same photographing parameter. For example, a preset photographing parameter or a photographing parameter for photographing the first scratch may be stored in a storage device, and when images of other scratches are photographed, the stored photographing parameter may be called for using. In some embodiments, a current photographing parameter may be recorded by setting a bar code, a two-dimensional code, or the like on the cultivation device, and then the photographing parameter may be obtained directly by scanning the code during a next photographing.

In some embodiments, a movement distance of each movement of the sample stage may be determined based on the total photographing length and the count of view fields of the scratch to realize the automatic photographing of the scratch. For example, assuming that the total photographing length of the scratch is 3 cm and the count of view fields is 10, a movement distance for each photographing is 0.3 cm. That is, every time the sample stage moves 0.3 cm in an extending direction of the scratch, the view field is photographed once.

In some embodiments, a plurality of scratches may be formed after the scratching operation is performed on the cells, and the cell image analysis device may photograph the plurality of scratches in sequence based on preset photographing parameters corresponding to the plurality of scratches. For example, a first scratch and a second scratch may be formed after the scratching operation is performed on the cells, and the cell image analysis device may automatically photograph the first scratch based on a preset photographing parameter corresponding to the first scratch and a position of the first scratch. The cell image analysis device may determine, based on a positional relationship between a first scratching gap corresponding to the first scratch and a second scratching gap corresponding to the second scratch, a positional relationship between the first scratch and the second scratch. The cell image analysis device may automatically photograph the second scratch based on the positional relationship between the first scratch and the second scratch, and the preset photographing parameter. For example, after the photographing the first scratch is completed, the movement direction and the movement distance of the sample stage may be controlled to photograph the second scratch based on a coordinate of a photographing end position of the first scratch and a coordinate of a photographing start position of the second scratch. The coordinate of the photographing end position of the first scratch and the coordinate of the photographing start position of the second scratch may be determined based on positions of the first scratching gap and the second scratching gap on the scratching portion.

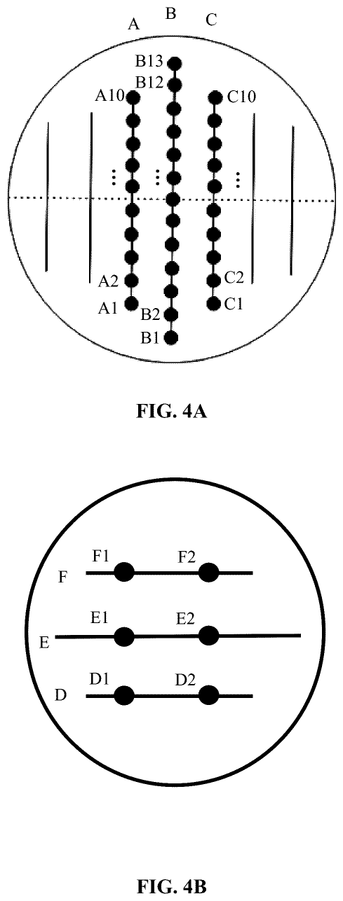

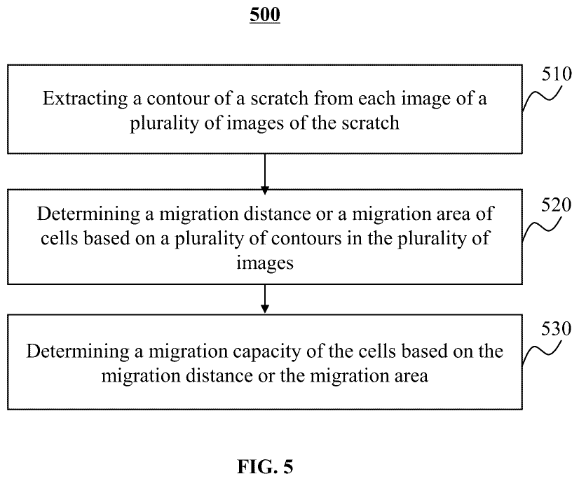

In some embodiments, in order to improve the efficiency of photographing the plurality of scratches, a more efficient photographing sequence may be determined based on positions of the plurality of scratches. For example, based on a coordinate of each photographing view field of each scratch of the plurality of scratches, an algorithm may automatically calculate the most efficient moving sequence of the photographing module or the sample stage, and the photographing module or the sample stage may be controlled to move in this moving sequence. Specifically, when photographing a plurality of scratches that are parallel to each other, it is possible to select a “ [symbol] ” shape to photograph. More description of the photographing sequence may be found in FIG. 4A and FIG. 4B and their related descriptions.

In step 250, the cell image analysis device 102 (or the processing device 103) may determine the migration capacity of the cells based on the plurality of images of the at least one scratch and the plurality of preset time points corresponding to the plurality of images.

The migration capacity of the cells refers to the ability of the cells to move from an initial position to another position. The migration capacity of the cells may be evaluated by a migration rate of the cells. The migration rate of the cells refers to a ratio of a migration area of the cells (or a migration distance) to a test duration.

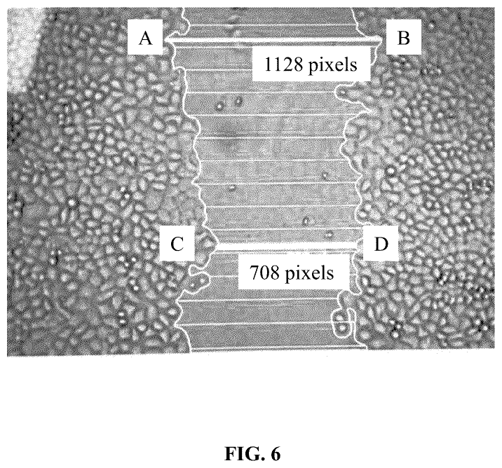

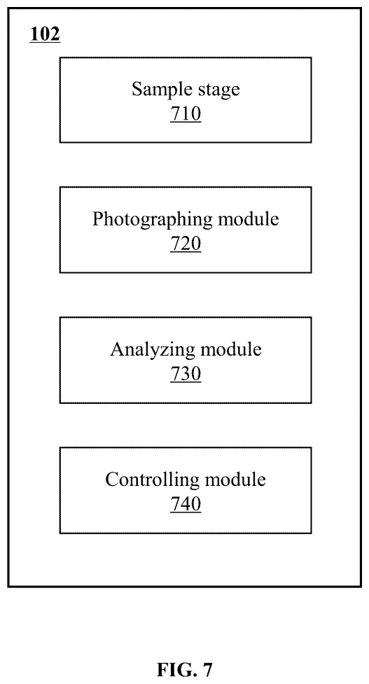

In some embodiments, the processing device 103 (or the cell image analysis device) may extract a contour of the at least one scratch from each of the plurality of images. The processing device 103 (or the cell image analysis device) may determine the migration distance or the migration area of the cells based on the contour. The processing device 103 (or cell image analysis device) may determine the migration capacity of the cells based on the migration distance or the migration area, and the plurality of preset time points. More description of the determination of the migration capacity of the cells can be found elsewhere in the present disclosure (e.g., FIG. 5 and FIG. 6 and their related descriptions).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, step 210 and/or step 230 may be omitted. As another example, steps 220 and 230 may be combined into a single step. As yet another example, in step 240, the cell image analysis device may automatically photograph the scratch based solely on the position of the scratch. Specifically, the position coordinates of the plurality of view fields of the scratch may be input into the cell image analysis device, and the cell analyzing device may automatically photograph and analyze the plurality of view fields based on the position coordinates of the plurality of view fields of the scratch. In some embodiments, before step 240, the cultivation medium in the cultivation portion needs to be aspirated, and a new cultivation medium is added after the photographing is completed. In some embodiments, instead of aspirating the cultivation medium in the cultivation portion, the scratches may be photographed and analyzed with the cultivation medium present. In such cases, after the cultivation portion is placed on the sample stage, it is necessary to wait for a liquid level of the cultivation medium to calm down before photographing in order to prevent fluctuations in the liquid level from affecting the accuracy of the photographing of the scratch.

In some embodiments, the at least one scratch may be formed by performing the scratching operation on the cells by a cell scratching device. In some embodiments, the scratching operation may be performed using a manual scratching device, a semi-automatic scratching device, or a fully automatic scratching device. For example, the scratching operation may be performed using the cell scratching device illustrated in FIG. 2A. As another example, the scratching operation may be performed using the cell scratching device illustrated in FIG. 8 to FIG. 16B. As yet another example, the cell scratching operation may also be performed using other means. The at least one scratch may then be photographed and analyzed by the cell image analysis device to determine the migration capacity of the cells. In some embodiments, the scratch may be photographed and analyzed using the cell image analysis device illustrated in FIGS. 2A to 7. In some embodiments, the scratch may be photographed and analyzed using other means. For example, the scratch may be photographed and analyzed using a microscope.

FIG. 2B is a flowchart illustrating an exemplary process for determining a cell migration capacity according to some embodiments of the present disclosure. In some embodiments, at least a portion of a process 205 may be performed by the cell scratching device 101, the cell image analysis device 102, or the processing device 103. For example, the process 205 may be stored as instructions (e.g., an application program) in a storage device (e.g., the storage device 104) and invoked and executed by the cell scratching device 101, the cell image analysis device 102, or the processing device 103. The operations of the processes shown below are for illustrative purposes only. In some embodiments, the process 205 may be accomplished utilizing one or more additional operations not described herein and/or without one or more of the operations described herein.

In step 260, a scratching operation is performed on cells through a cell scratching device (e.g., the scratching portion 101-2) to form at least one scratch.

More description of the cell scratching device and the scratching operation using the cell scratching device may be found in step 220 in FIG. 2A, FIG. 8 to FIG. 16B, and their related descriptions.

In step 270, the migration capacity of the cells is determined by photographing and analyzing the at least one scratch by the cell image analysis device 102 (or the processing device 103).

In some embodiments, at least one image of the at least one scratch may be obtained by positioning and photographing the at least one scratch through the cell image analysis device 102. The cell image analysis device 102 may determine the migration capacity of the cells based on the at least one image of the at least one scratch. More description of photographing and analyzing the at least one scratch to determine the migration capacity of the cells may be found in the related description of steps 240 and 250 in FIG. 2A.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

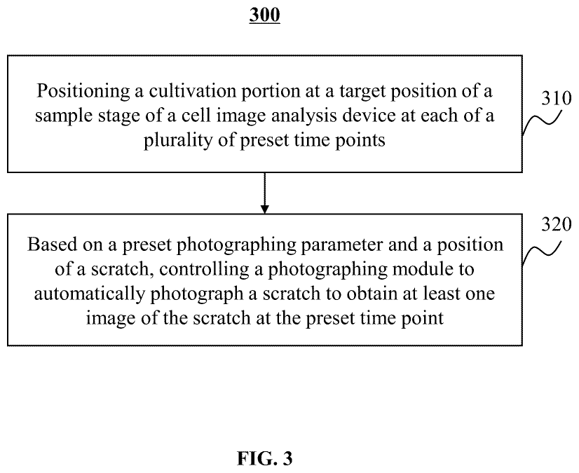

FIG. 3 is a flowchart illustrating an exemplary process for obtaining a scratching image according to some embodiments of the present disclosure. In some embodiments, at least a portion of a process 300 may be performed by the cell image analysis device 102 or the processing device 103. For example, the process 300 may be stored in the form of instructions (e.g., an application program) in a storage device (e.g., the storage device 104) and invoked and/or executed by the cell image analysis device 102 or the processing device 103. The operations of the processes shown below are for illustrative purposes only. In some embodiments, the process 300 may be accomplished utilizing one or more additional operations that are not described herein and/or without one or more of the operations described herein. Additionally, an order of the operations of process 300 illustrated in FIG. 3 and described below is not intended to be limiting.

In step 310, a cultivation portion is positioned at a target position of a sample stage (e.g., the sample stage 710) of the cell image analysis device at each of a plurality of preset time points.

The target position refers to a position on the sample stage for placing the cultivation portion. For example, the target position may be a center position of the sample stage.

In some embodiments, a manual positioning may be performed by designing a base point or a baseline on the cultivation portion and the sample stage. For example, cross-orientation digital marks may be provided on both the cultivation portion and the sample stage. By aligning the corresponding cross-orientation digital marks on the cultivation portion and the sample stage, the positioning of the cultivation portion on the sample stage may be accomplished. In some embodiments, a cultivation portion adapter may be provided on the sample stage to enable the positioning of the cultivation portion on the sample stage. For example, a recessed adapter is provided on a bottom of the cultivation portion, and a correspondingly protruding adapter is provided on a surface of the sample stage, and the positioning of the cultivation portion is realized by matching the two adapters.

In some embodiments, since the cultivation portion is a symmetrical structure (e.g., a round petri dish), an orientation marker may be provided at a non-center position of the cultivation portion, and different cultivation portions have their own markers. A position and orientation of each cultivation portion placed on the sample stage may be automatically recorded based on the marker and the orientation marker of the cultivation portion. The cultivation portion may be positioned again at different preset time points based on a position and orientation of the cultivation portion placed on the sample stage at a first preset time point to ensure that positions and orientations of the cultivation portion placed on the sample stage are consistent at the different preset time points.

In some embodiments, a cultivation portion moving mechanism may be provided on the sample stage, and the moving mechanism may move the cultivation portion to the target position. For example, after the cultivation portion is placed on the sample stage, the cell image analysis device may photograph the cultivation portion (e.g., the cultivation portion is photographed at a low magnification) to obtain an image of the cultivation portion. By analyzing the image of the cultivation portion, a center of the cultivation portion may be determined. Based on a positional relationship between the center of the cultivation portion and a zero position of the sample stage, a movement direction and a movement distance of the cultivation portion are determined and the cultivation portion moving mechanism is controlled to move the cultivation portion.

In some embodiments, for a more accurate positioning, baselines (e.g., a straight line passing through a center of the petri dish is a baseline) may be provided on the cultivation portion and the sample stage. After obtaining the image of the cultivation portion, the baseline of the cultivation portion may be extracted from the image of the cultivation portion, and the movement direction and the movement distance of the cultivation portion may be determined based on a positional relationship between the baseline of the cultivation portion and the baseline of the sample stage, and the cultivation portion moving mechanism is controlled to move the cultivation portion.

In some embodiments, prior to photographing a scratch at each preset time point, or prior to photographing a plurality of scratches at a single preset time point, it is necessary to calibrate the position of the cultivation portion on the sample stage to ensure that the cultivation portion is at the same target position on the sample stage when being photographed each time. For example, corresponding base points (or baselines) may be set on both the cultivation portion and the sample stage, and whether the position of the cultivation portion on the sample stage needs to be calibrated may be determined by determining whether there has been a movement of the base points (or baselines) between the cultivation portion and the sample stage and a calibration manner (a direction and a distance in which the cultivation portion needs to be moved) may be determined as well.

In step 320, based on a preset photographing parameter and a position of a scratch, the cell image analysis device 102 (e.g., a control module 740 or the processing device 103) may control a photographing module (e.g., a photographing module 720) to automatically photograph the scratch to obtain at least one image of the scratch at the preset time point.

In some embodiments, since the present disclosure uses the cell scratching device instead of a manual scratching, the position of the formed scratch corresponds to a position of a scratching gap in the cell scratching device, and thus, the position of the scratching gap in the cell scratching device may be obtained to determine scratch information, and a plurality of scratches may be photographed sequentially based on the scratch information. The scratch information includes various information related to the scratch, for example, a scratch interval, the position of the scratch, a positional relationship between an endpoint of the scratch and the scratch, or the like. For example, the cell image analysis device may obtain a position of a scratching gap corresponding to the scratch from the cell scratching device and determine the position of the scratch based on the position of the scratching gap, and further photograph the scratch. As another example, a user may input information about a position of a scratching gap corresponding to the scratch into the cell image analysis device, and the cell image analysis device may determine the position of the scratch based on the position of the scratching gap, and further photograph the scratch.

In some embodiments, the scratching gaps of the cell scratching device may be numbered, and information (a length, a width, a positional coordinate) of scratching gaps may be stored based on numbers of the scratching gaps. When a scratch produced by a certain scratching gap is required to be photographed, information of the scratching gap may be directly obtained as the scratch information.

In some embodiments, the scratch information may be determined by manners such as labeling the endpoint of the scratch, obtaining an image of the cultivation portion, or based on a light transmission degree of cells, or the like. For example, the endpoint of the scratch may be fluorescently labeled, and the endpoint of the scratch may be determined by fluorescent tracking. As another example, due to a difference in light transmission degrees between a scratched region and a non-scratched region (the non-scratched region is covered by cells and has a lower light transmission degree than that of the scratched region), a light of an appropriate intensity (e.g., a white light that does not affect cell growth) may be applied to the bottom of the cultivation portion, and a region with a better light transmission is the scratched region and a region with a poorer light transmission is the non-scratched region, and a length of the scratch and intervals between different scratches are recorded. As yet another example, the cultivation portion may be photographed by low magnification to obtain the image of the cultivation portion, and the scratch information may be determined by identifying information of a scratch in the image of the cultivation portion. Specifically, the photographing module of the cell image analysis device may include at least one local camera and at least one global camera, the global camera may photograph a global image of the scratch and determine the scratch information based on the global image, and the local camera may photograph images of a plurality of view fields of the scratch.

In some embodiments, the scratch may be positioned by a mechanical device. For example, a position corresponding to the scratch on the bottom of the cultivation portion is provided with the recessed adapter for positioning the scratch, and a bottom of the sample stage is provided with a protruding adapter corresponding to the recessed adapter, and the positioning of the scratch is achieved by matching the two adapters.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

FIG. 4A is a schematic diagram illustrating photographing along a direction parallel to an extending direction of a scratch according to some embodiments of the present disclosure. FIG. 4B is a schematic diagram illustrating photographing along a direction perpendicular to an extending direction of a scratch according to some embodiments of the present disclosure. Black dots in the figures indicate photographing view fields.

As shown in FIG. 4A, a scratch A includes 10 photographing view fields, A1, A2, . . . , and A10; a scratch B includes 13 photographing view fields, B1, B2, . . . , and B13; a scratch C includes 10 photographing view fields, C1, C2, . . . , and C10. Extending directions of the scratch A, the scratch B, and the scratch C are parallel to each other. In some embodiments, a cell image analysis device may control a sample stage (or a photographing module) to move along a direction parallel to the extending direction of a scratch in order to obtain a plurality of images of multiple view fields of the scratch along the extending direction. For example, the cell image analysis device may control the photographing module (e.g., a camera) to move to a starting photographing position (e.g., a photographing view field A1) of the scratch A and control the photographing module to move along the direction parallel to the extending direction of the scratch A to photograph A1, A2, . . . , A10 sequentially, until the photographing of the scratch A is completed, so as to realize the observation and analysis of different view fields of a single scratch.

Further, the cell image analysis device may control the photographing module to move to a starting photographing position (e.g., a photographing view field B1) of the scratch B, and control the photographing module to move along the direction parallel to the extending direction of the scratch B to photograph B1, B2, . . . , B10 sequentially, until the photographing of the scratch B is completed. Finally, the cell image analysis device may control the photographing module to move to a starting photographing position (e.g., a photographing view field C1) of the scratch C, and control the photographing module to move along the direction parallel to the extending direction of the scratch C to photograph C1, C2, . . . , C10 sequentially, until the photographing of the scratch C is completed.

In some embodiments, in order to improve the photographing efficiency, after completing the photographing of the scratch A, the photographing module may move to the photographing view field B13 of the scratch B, and photograph B13, B12, . . . , B1 sequentially until the photographing of the scratch B is completed. Further, the photographing module may move to the photographing view field C1 of the scratch C, and photograph C1, C2, . . . , C10 sequentially until the photographing of the scratch C is completed.

As shown in FIG. 4B, a scratch D, a scratch E, and a scratch F have two photographing view fields, respectively. Extending directions of the scratch D, the scratch E, and the scratch F are parallel to each other. In some embodiments, the cell image analysis device may control the sample stage (or the photographing module) to move along a direction perpendicular to the extending direction of the scratch to obtain a plurality of images of a plurality of scratches. For example, the cell image analysis device may control the photographing module (e.g., the camera) to move to a starting photographing position (e.g., a photographing view field D1) of the scratch D and control the photographing module to move along the direction perpendicular to the extending direction of the scratch D, and sequentially photograph the photographing view field D1 of the scratch D, a photographing view field E1 of the scratch E, and a photographing view field F1 of the scratch F, so as to realize the comparative observation and analysis of the plurality of scratches.

Further, the cell image analysis device may control the photographing module to move to a photographing view field D2 of the scratch D, and control the photographing module to move along the direction perpendicular to the extending direction of the scratch D, and sequentially photograph the photographing view field D2 of the scratch D, a photographing view field E2 of the scratch E, and a photographing view field F2 of the scratch F. In some embodiments, in order to improve the efficiency of the photographing, after completing the photographing of the photographing view field F1 of the scratch F, the photographing module may be moved to the photographing view field F2 of the scratch F, and the photographing module may sequentially photograph the photographing view field F2 of the scratch F, the photographing view field E2 of the scratch E, and the photographing view field D2 of the scratch D.

FIG. 5 is a flowchart illustrating an exemplary process for determining a cell migration capacity according to some embodiments of the present disclosure. In some embodiments, at least a portion of a process 500 may be performed by the cell image analysis device 102 or the processing device 103. For example, the process 500 may be stored in the form of instructions (e.g., an application program) in a storage device (e.g., the storage device 104) and invoked and/or executed by the cell image analysis device 102 or the processing device 103. The operations of the processes shown below are for illustrative purposes only. In some embodiments, the process 500 may be accomplished utilizing one or more additional operations that are not described herein and/or without one or more of the operations described herein. Additionally, an order of the operations of process 500 illustrated in FIG. 5 and described below is not intended to be limiting.

In step 510, the cell image analysis device 102 (e.g., an analyzing module 730 or the processing device 103) may extract a contour of a scratch from each image of a plurality of images of the scratch.

In some embodiments, the plurality of images of the scratch is obtained by photographing the scratch at a plurality of preset time points. For example, the plurality of images of the scratch may be obtained by photographing the scratch at a preset time point of 0 hours, 2 hours, and 4 hours, respectively. In some embodiments, the processing device 103 (or the cell image analysis device) may extract the contour of the scratch from the image based on an image segmentation algorithm. Exemplary image segmentation algorithm may include a region-based algorithm (e.g., a threshold segmentation, a region growth segmentation), an edge detection segmentation algorithm, a compression-based algorithm, a histogram-based algorithm, a dual clustering algorithm, etc.

In step 520, the cell image analysis device 102 (e.g., the analyzing module 730 or the processing device 103) may determine a migration distance or a migration area of cells based on a plurality of contours in the plurality of images.

The migration distance of the cells refers to a difference of distances of lines connecting cells at two sides of the scratch at different preset time points. For example, a difference of distances of lines connecting cells at two sides of the scratch in images obtained at different preset time points may be determined as a migration distance of the cells during that time range. Specifically, assuming that a distance of a line connecting middle cells at two sides of a contour of the scratch in an image obtained at the preset time point of 0 hour is 1.50 mm, and a distance of a line connecting middle cells at two sides of a contour of the scratch in an image obtained at the preset time point of 2 hours is 1.20 mm, and then it may be determined that a migration distance of the cells is 0.3 mm in two hours.

In some embodiments, a plurality of migration distances corresponding to a plurality of positions of the scratch may be determined based on the contour of the scratch. The migration distance may include a maximum migration distance, a minimum migration distance, an average migration distance, or the like.

The migration area of the cells refers to a difference of areas of the scratch at different time points. For example, a difference of areas of contours of the scratch in the images obtained at different preset time points may be determined as a migration area during that time range. Specifically, assuming that an area of a contour of the scratch in the image obtained at the preset time point of 2 hours is 20 mm², and an area of a contour of the scratch in an image obtained at the preset time point of 4 hours changes to 15 mm², then it may be determined that a migration area of the cells is 5 mm² in two hours.

FIG. 6 is a schematic diagram illustrating an exemplary scratching image according to some embodiments of the present disclosure. White lines on two sides of a scratched region in FIG. 6 indicate a scratching width. As shown in FIG. 6, a maximum scratching width AB (i.e., a maximum distance between lines connecting cells on both sides of the contour) is 1128 pixels, a minimum scratching width CD (i.e., a minimum distance between the lines connecting cells on both sides of the contour) is 856 pixels, and an average scratching width (i.e., an average distance between the lines connecting cells on both sides of the contour) is 708 pixels, a scratching area is 1641960 pixels, and a photographing time (i.e., a time difference between a time when a scratching operation was completed and a time when a scratching image was photographed) is 2216 ms.

Step 530, the cell image analysis device 102 (e.g., the analyzing module 730 or the processing device 103) may determine a migration capacity of the cells based on the migration distance or the migration area.

In some embodiments, the migration capacity of the cells may be determined based on the migration distance or the migration area, and a plurality of preset time points corresponding to a plurality of images. For example, a ratio (i.e., a migration rate) of the migration distance (or the migration area) to a time taken to generate the migration distance (or the migration area) may be determined as the migration capacity of the cells. Specifically, assuming that the migration area of the cells is 5 mm² in two hours, then the migration capacity of the cells may be determined to be 2.5 mm²/h.

In some embodiments, a plurality of photographing view fields of a plurality of scratches of cells may be analyzed at different preset time points to determine a plurality of migration rates of the cells corresponding to the different preset time points, different scratches, and different photographing view fields, and an average value of the plurality of migration rates may be determined as the migration capacity of the cells.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

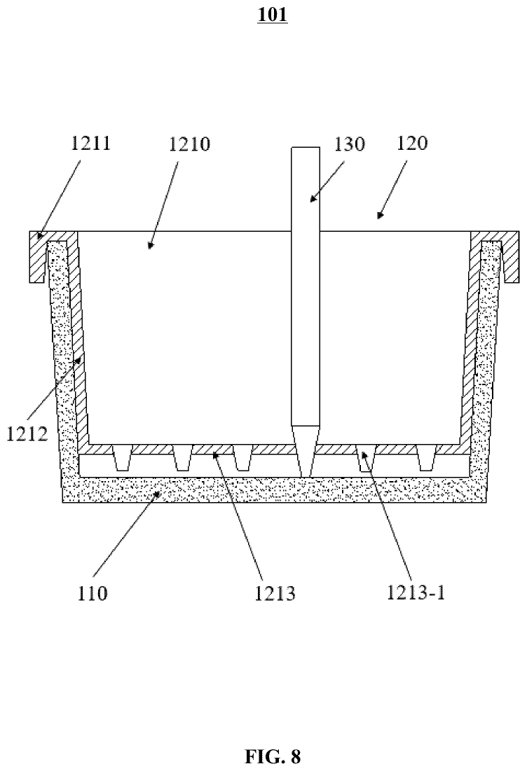

FIG. 7 is a block diagram illustrating an exemplary cell image analysis device according to some embodiments of the present disclosure. As shown in FIG. 7, the cell image analysis device 102 may include the sample stage 710, the photographing module 720, the analyzing module 730, and the controlling module 740.

The sample stage 710 may be configured to accommodate a cell cultivation device (e.g., the cultivation portion 101-1). In some embodiments, the sample stage 710 may be provided with a base point (or a baseline), an adapter, and/or a moving mechanism for positioning the cell cultivation device. More description of the positioning of the cell cultivation device on the sample stage may be found in FIG. 3 and its descriptions.

The photographing module 720 may be configured to photograph an image. In some embodiments, the photographing module 720 may be and/or include any suitable device capable of obtaining image data. For example, the photographing module 720 may include a spherical camera, a dome camera, etc. In some embodiments, the photographing module 720 may include a black and white camera, a color camera, an infrared camera, or the like. In some embodiments, the photographing module 720 may include a digital camera, an analog camera, etc. In some embodiments, the photographing module 720 may include a monocular camera, a binocular camera, a multicamera, or the like. In some embodiments, the photographing module 720 may photograph a scratch to obtain an image of the scratch. In some embodiments, the photographing module 720 may photograph the cultivation portion to obtain an image of the cultivation portion.

The analyzing module 730 may be configured to analyze data. In some embodiments, the analyzing module 730 may determine a migration capacity of cells based on the image of the scratch. For example, the analyzing module 730 may extract a contour of the scratch in the image. The analyzing module 730 may determine a migration distance or a migration area of the cells based on the contour of the scratch. The analyzing module 730 may determine the migration capacity of the cells based on the migration distance or the migration area. More description of determining the migration capacity of the cells may be found in FIGS. 2A, 5-6 and their descriptions.

The controlling module 740 may be configured to control other components in cell image analysis device 102. For example, the controlling module 740 may control the movement of the sample stage. As another example, the controlling module 740 may control a photographing module to photograph the scratch.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the analyzing module 730 and/or the controlling module 740 may be integrated into one module. As another example, the analyzing module 730 and/or the controlling module 740 may be omitted. The processing device 103 may be configured to analyze data and control other components in cell image analysis device 102.

The cell scratching device 101 in the embodiments of the present disclosure would be described in detail below in conjunction with FIG. 8 to FIG. 16B. It should be noted that the following embodiments are for the sole purpose of explaining the present disclosure and do not constitute a limitation of the present disclosure.

FIG. 8 is a schematic diagram illustrating an exemplary cross-section of a cell scratching device according to some embodiments of the present disclosure. As shown in FIG. 8, in some embodiments, the cell scratching device 101 may include the cultivation portion 110 and the scratching portion 120. The cultivation portion 110 may be configured for cell cultivation, and the scratching portion 120 may be configured to realize cell scratching.

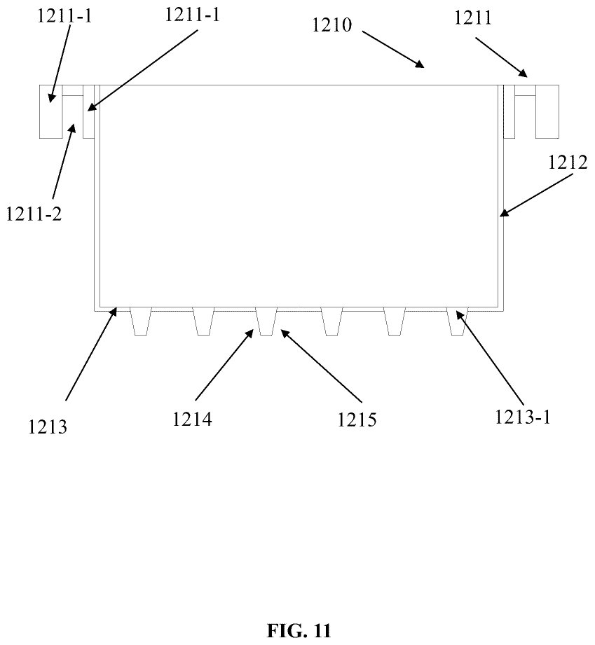

In some embodiments, the cultivation portion 110 may include a petri dish or a cultivation plate. The petri dish may be made of glass or plastic. In some embodiments, the cultivation portion 110 may include a D90 petri dish, a six-well cultivation plate (shown in FIG. 10), or the like.

In some embodiments, the scratching portion 120 may include a scratching cover plate 1210 and the scratching member 130. In some embodiments, the scratching cover plate 1210 may include a base plate 1213, a connecting member 1212, and a position-limiting structure 1211 that are sequentially connected. The base plate 1213 and the position-limiting structure 1211 are disposed at two ends of the connecting member 1212.

As shown in FIG. 8, in some embodiments, when a scratching operation is performed, the scratching cover plate 1210 may be mounted and secured to the cultivation portion 110 by the position-limiting structure 1211, henceforth avoiding loosening between the cultivation portion 110 and the scratching portion 120 and affecting the scratch. In some embodiments, when the scratching portion 120 is mounted and secured to the cultivation portion 110 by the position-limiting structure 1211, an interval between the base plate 1213 of the scratching cover plate 1210 and a bottom of the cultivation portion 110 may be in a range of 0.5 to 1.5 mm. By reserving a certain distance between the base plate of the scratching cover plate and the bottom of the cultivation portion, it is possible to prevent the scratching cover plate from pressing cells in the cultivation portion. In some embodiments, the position-limiting structure 1211 may include at least one of an inverted U-shaped groove, a flap protrusion, a flap groove, a rubber catch, a threaded structure, or a mortise-and-tenon structure, etc., and the position-limiting structure 1211 matches with a sidewall of the cultivation portion 110. For example, as shown in FIG. 8, when the position-limiting structure 1211 is a flap groove structure, a width of a groove matches a thickness of the sidewall of the cultivation portion 110. By matching the position-limiting structure 1211 with the sidewall of the cultivation portion 110, it is possible that there is no gap between the scratching cover plate and the cultivation portion when they are fitted together, and that the scratching cover plate does not deflect when the scratching operation is performed, which is able to ensure that the scratch is straight and uniform.

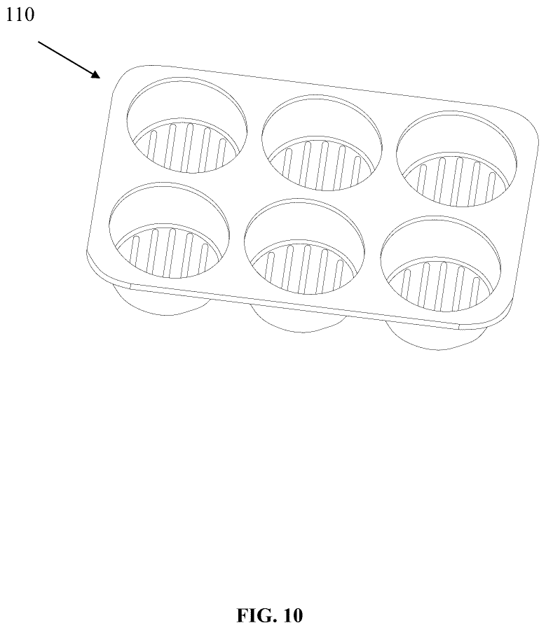

In some embodiments, the base plate 1213 may be provided with a plurality of scratching gaps 1213-1, the plurality of scratching gaps are distributed parallel to each other and evenly spaced apart. As shown in FIG. 8, in some embodiments, when the scratching operation is performed, a tip of the scratching member 130 may be inserted into one of the scratching gaps 1213-1, and by moving along one end of the scratching gaps 1213-1 to the other end of the scratching gaps 1213-1, the scratching operation on cells is achieved. In some embodiments, the base plate 1213 may be provided with 6 to 15 scratching gaps 1213-1, and an interval between any two adjacent scratching gaps may be in a range of 0.5 cm to 1 cm. For example, the base plate 1213 may be provided with seven scratching gaps, and an interval between two adjacent scratching gaps may be 1 cm. As another example, as shown in FIG. 9, the base plate 1213 may be provided with 14 scratching gaps, and an interval between two adjacent scratching gaps may be 0.5 cm. In some embodiments, scratching gaps of different counts, different shapes/different widths may be provided on the base plate 1213, depending on a structure of the cultivation portion 110 and/or a need of a cell scratching experiment. For example, for a six-well petri dish, scratching gaps of a corresponding scratching cover plate thereof may be 3 or 5, and an interval between two adjacent scratching gaps may be 0.5 cm or 1 cm.

In some embodiments, a cross-section of each of the scratching gaps 1231-1 may be a trapezoidal shape that is wide at a top and narrow at a bottom, the trapezoidal shape may be shaped to match a tip shape of the scratching member 130, so that when the tip of the scratching member 130 is inserted into the scratching gap to the bottom, it is just in contacts with the cells in the cultivation portion 110. In some embodiments, the scratching cover plate 1210 is removably coupled to the scratching member 130.

In some embodiments, a length of the connecting member 1212 may be a predetermined length. Generally, a user may design a length of a corresponding connecting member 1212 based on a dimension of an actual cultivation portion 110 and other relevant requirements (e.g., an interval between the base plate 1213 and the bottom of the cultivation portion 110 after the scratching cover plate 1210 is installed, etc.). Therefore, after the scratching cover plate 1210 is mounted and fixed to the cultivation portion 110 by the position-limiting structure 1211, the interval between the base plate 1213 and the bottom of the cultivation portion 110 may be in a range of 0.5 mm to 1.5 mm. Preferably, the interval between the base plate 1213 and the bottom of the cultivation portion 110 may be 1 mm.

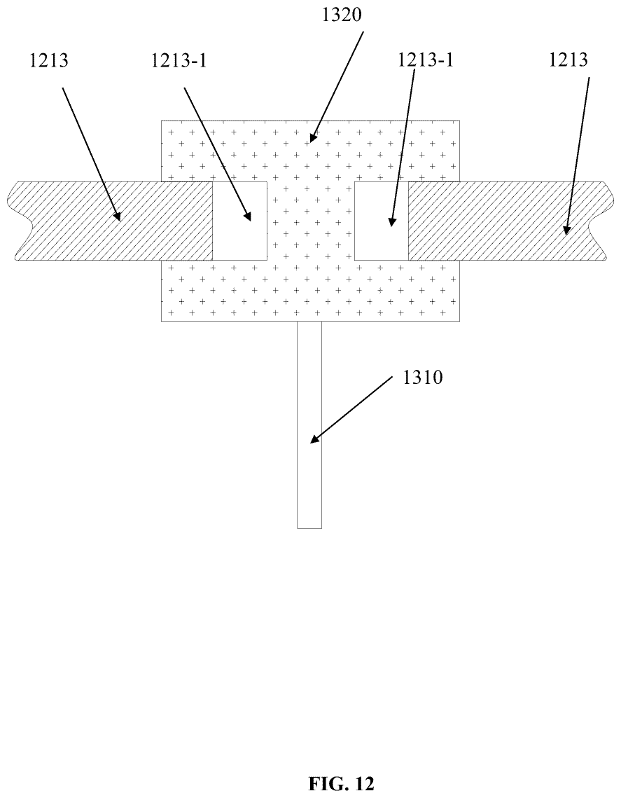

Referring to FIG. 11, FIG. 11 is a schematic diagram illustrating an exemplary structure of the scratching cover plate 1210 according to some embodiments of the present disclosure.

In some embodiments, the position-limiting structure 1211 may be set as an inverted U-shaped groove. As shown in FIG. 11, the position-limiting structure 1211 may include two fixing members 1211-1, one of the two fixing members 1211-1 is coupled to the connecting member 1212, and a position-limiting groove 1211-2 is formed between the two fixing members 1211-1, and a width of the position-limiting groove 1211-2 matches with the thickness of the sidewall of the cultivation portion 110.

In other embodiments, the position-limiting structure 1211 may include only a fixing member 1211-1, and the position-limiting groove 1211-2 is formed between the fixing member 1211-1 and the connecting member 1212.

In some embodiments, the fixing member 1211-1 may be a flap of the connecting member 1212.

In other embodiments, the fixing member 1211-1 may be a rubber catch. The rubber catch has a greater friction with the sidewall of the cultivation portion 110 and is less prone to a relative displacement, and thus the installation of the scratching cover plate 1210 is more stable and less prone to loosening. In some embodiments, a surface of the rubber catch near a side of the connecting member 1212 may be provided with a texture such as bumps to improve the stability of the position-limiting, or the like.

In other embodiments, internal threads may also be provided in the position-limiting groove 1211-2, and corresponding external threads (not shown in the figures) are provided on an outer sidewall of the cultivation portion 110, and the scratching cover plate 1210 is threadedly connected to the cultivation portion 110. Setting a threaded connection not only makes the installation of the scratching cover plate 1210 more stable but also makes the scratching cover plate 1210 rotate while moving in a length direction of the threads. The threaded connection can therefore make the interval between the base plate 1213 and the bottom of the cultivation portion 110 adjustable, which is convenient for experimentalists to operate. The experimentalists can adjust the interval between the base plate 1213 and the bottom of the cultivation portion 110 according to different models of the cultivation portion 110 and the scratching member 130, henceforth enhancing the scratching effect.

In other embodiments, the position-limiting structure 1211 may also be connected to the cultivation portion 110 using a mortise-and-tenon structure, i.e., a snap fit. A groove is provided on an inner wall of the position-limiting groove 1211-2, and a corresponding protrusion (not shown in the figure) is provided in the outer sidewall of the cultivation portion 110, and the fixation of the scratching cover plate 1210 is realized by a cooperation between the groove and the protrusion. In other embodiments, the outer sidewall of the cultivation portion 110 may be provided with a plurality of corresponding protrusions along a height direction, and the interval between the base plate 1213 and the bottom of the cultivation portion 110 may be changed through a snap-fit between the groove and protrusions at different height positions. Thus, the experimenters may adjust the interval between the base plate 1213 and the bottom of the cultivation portion 110 according to different models of the cultivation portion 110 and the scratching member 130 to enhance the scratching effect.

In some embodiments, the scratching member 130 may include a scratching needle 1310. Two sides of the scratching gap 1213-1 are provided with a first position-limiting member 1214 and a second position-limiting member 1215, respectively. In some embodiments, the first position-limiting member 1214 and the second position-limiting member 1215 may both be located on a side of the base plate 1213 near the bottom of cultivation portion 110. An interval between the first position-limiting member 1214 and the second position-limiting member 1215 decreases in a direction from the base plate 1213 to the bottom of the cultivation portion 110. Therefore, an inverted conical shape with a large upper opening and a small lower opening is presented between the first position-limiting member 1214 and the second position-limiting member 1215, which is able to catch the scratching needle 1310 when the scratching needle 1310 is inserted into the scratching gap, thereby facilitating the limitation of the scratch needle 1310 and ensuring that a length of the scratching needle 1310 extending out of the base plate 1213 remains unchanged, and accordingly, enhancing the stability of the scratching. In some embodiments, a width of a lower opening between the first position-limiting member 1214 and the second position-limiting member 1215 is less than a width of the scratching gap 1213-1, i.e., a distance of a side of a gap between the first position-limiting member 1214 and the second position-limiting member 1215 is less than the width of the scratching gap 1213-1. The side of the gap is close to the bottom of the cultivation portion 110.

In some embodiments, the scratching member 130 may also include a slider 1320, and the scratching needle 1310 may be mounted to the slider 1320. When the scratching operation is performed, the slider 1320 may slide using the scratching gap 1213-1 as a guide rail to drive the scratching needle 1310 from one end of one of the scratching gaps to the other end of the scratching gap to achieve the scratching operation. Because the slider 1320 and the scratching gap 1213-1 as a guide rail are tightly fitted and not easily deflected, the scratching needle 1310 may be better perpendicular to the bottom of the cultivation portion 110 with a high stability degree, thus guaranteeing the uniformity and stability of the scratching effect.

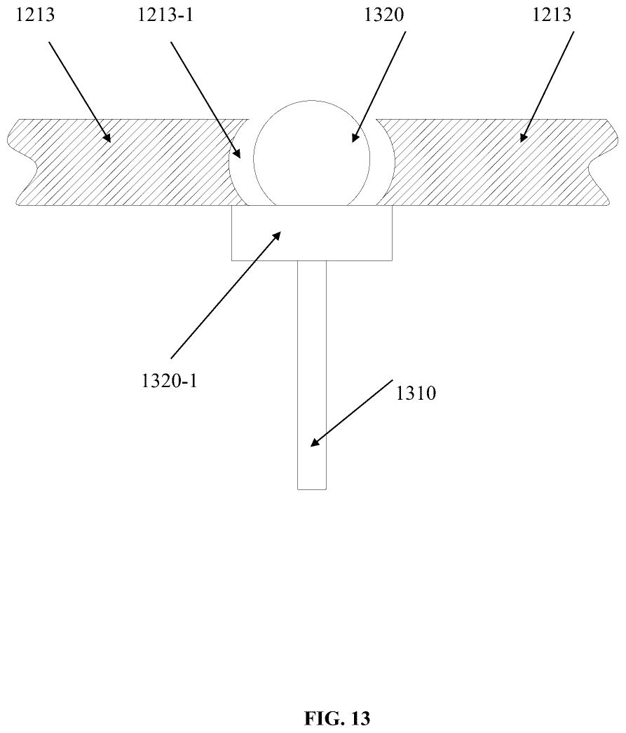

Referring to FIG. 12, FIG. 12 is a schematic diagram illustrating an exemplary matching structure of the scratching member 130 and the scratching gap 1213-1 according to some embodiments of the present disclosure.

In some embodiments, the slider 1320 may be provided with a threaded hole, the threaded hole may be provided perpendicularly to the base plate 1213, and an outer wall of the scratching needle 1310 may be provided with external threads (not shown in the figures) corresponding to the threaded hole, thereby allowing the scratching needle 1310 to threadedly connected to the slider 1320. Setting a threaded connection strengthens the installation stability of the scratching needle 1310 on the one hand, and allows the scratching needle 1310 to adjust a length of the slider 1320 that extends out of the slider 1320 during rotation on the other hand. Thus, when the interval between the base plate 1213 and the bottom of the cultivation portion 110 is too large or too small, it is possible to ensure that a tip of the scratching needle 1310 can contact the bottom of the cultivation portion 110 by adjusting the length of the scratching needle 1310 that extends out of the slider 1320, which facilitates the scratching operation. Furthermore, a handle (not shown in the figure) may be provided on a top of the slider 1320 to facilitate an operation by an experimenter.

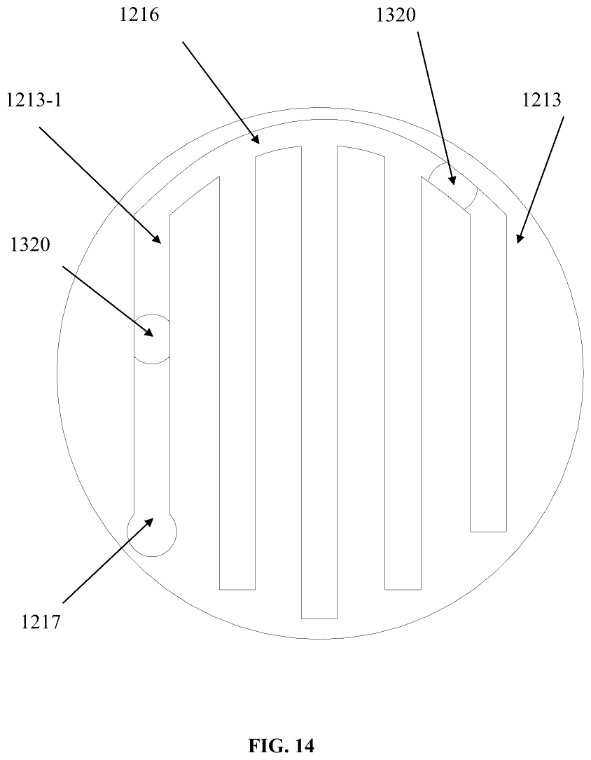

Referring to FIG. 13, FIG. 13 is a schematic diagram illustrating another exemplary matching structure of the scratching member 130 and the scratching gap 1213-1 according to some embodiments of the present disclosure.

In some embodiments, as shown in FIG. 13, the slider 1320 may be spherical, and an interior of the scratching gap 1213-1 may be shaped to match the spherical slider. Setting the spherical slider can reduce a probability of the slider 1320 getting stuck in a corner to some extent. Further, in order to avoid the spherical slider itself from undergoing a roll that would cause the scratching needle 1310 to become unstable, other restriction structures may be additionally added, for example, a restriction structure 1320-1 is provided at a position on a bottom of the spherical slider exposed from the scratching gap 1213-1, so that the spherical slider itself does not roll. In other embodiments, the restriction structure 1320-1 may also be provided at a top of the spherical slider, which serves as a handle while providing a position-limiting function.

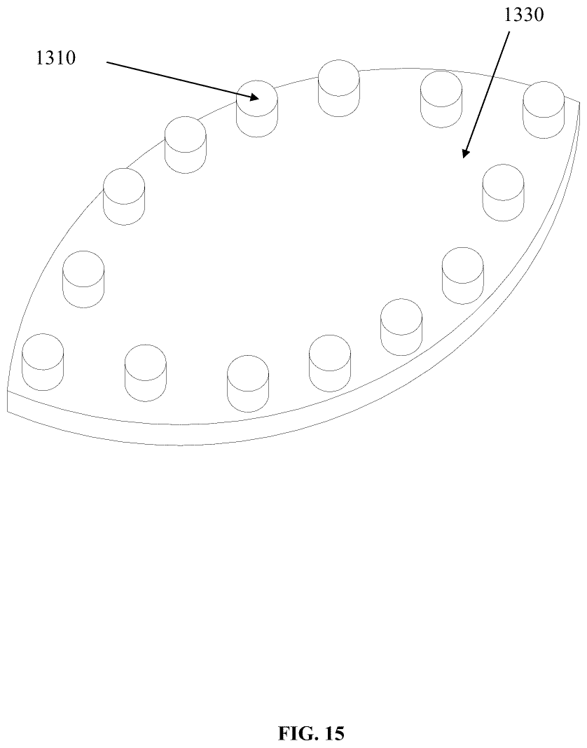

Referring to FIG. 14, FIG. 14 is a schematic diagram illustrating an exemplary structure of the base plate 1213 according to some embodiments of the present disclosure.

As shown in FIG. 14, in other embodiments, ends of a plurality of scratching gaps 1213-1 on a same side may be connected by a connection path 1216. As a result, the same slider 1320 may enter into the plurality of scratching gaps 1213-1, thereby reducing a count of scratching members 130, and accordingly, avoiding the impact on the scratching effect due to differences between different scratching members 130, thereby improving the stability and uniformity of the scratching.

Furthermore, as shown in FIG. 14, in some embodiments, the scratching gap 1213-1 may also be provided with an entry hole 1217. In some embodiments, a size of the entry hole 1217 is not smaller than a size of the slider 1320, so that the slider 1320 may enter the scratching gap 1213-1 through the entry hole 1217, thereby realizing a removable connection between the scratching member 130 and the base plate 1213, and facilitating the experimentalist to use a scratching member 130 of a corresponding specification to perform the scratching operation. Setting the entry hole 1217 is equivalent to one end of the guide rail (i.e., the scratching gap 1213-1) being left unclosed, and thus the slider 1320 may easily enter and exit the guide rail (i.e., the scratching gap 1213-1).

In other embodiments, the scratching member 130 may further include a mounting body 1330, a plurality of sets of scratching needles 1310 may be secured to the mounting body 1330, each set of scratching needles 1310 may include one or more scratching needles 1310, and each set of scratching needles 1310 corresponds to a scratching gap 1213-1.

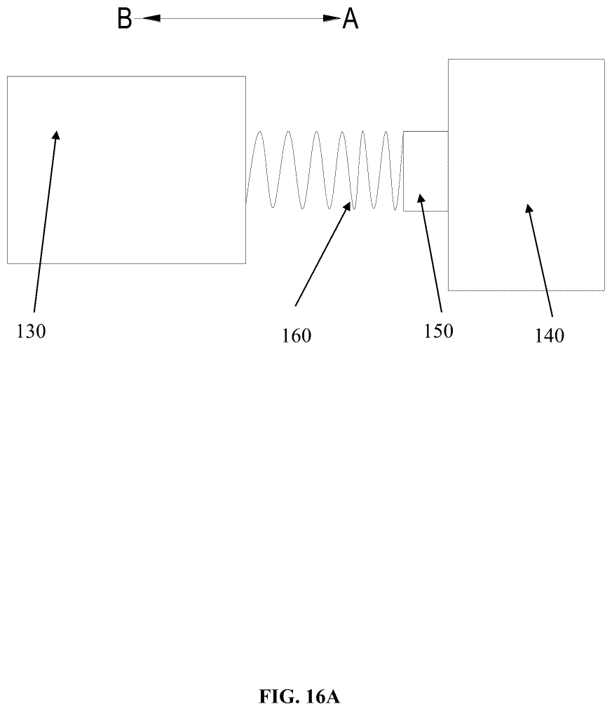

Referring to FIG. 15, FIG. 15 is a schematic diagram illustrating an exemplary structure of the scratching member 130 according to some embodiments of the present disclosure.

In some embodiments, a distribution of positions of a plurality of scratching needles 1310 may form two curves, each curve matches a shape of a line (e.g., the connecting path 1216) connecting ends of the plurality of scratching gaps 1213-1. When performing the scratching operation, one curve (which may be considered a starting curve) of the two curves may first be overlapped with the line connecting the ends of the plurality of scratching gaps 1213-1, where the scratching needles 1310 on the starting curve all located at a first end of the scratching gaps 1213-1, and the scratching needles 1310 on the other curve (which may be considered a termination curve) are located in a middle of the scratching gaps 1213-1. When the scratching reaches an end point, the scratching needles 1310 on the termination curve are all located at a second end of the scratching gaps 1213-1, and the scratching needles 1310 on the starting curve are located in the middle of the scratching gaps 1213-1. Specifically, for a set of scratching needles 1310 corresponding to a shortest scratching gap 1213-1, only one scratching needle 1310 may be included.

In some embodiments, the scratching portion 120 may further comprise a first magnet, and the scratching member 130 is provided with a second magnet that matches the first magnet. In some embodiments, the first magnet may be provided on the base plate 1213. In some embodiments, the second magnet may be the slider 1320 or other structures attached to the scratching needle 1310. When the scratching operation is performed, the first magnet may be moved, which attracts or repels the second magnet to move, which in turn drives the scratching needle 1310 to move to scratch. For example, the first magnet repels the second magnet, and the experimenter may manually move the first magnet along the scratching gap 1213-1, thereby driving the second magnet to drive the scratching needle 1310 to scratch. Since the first magnet is always close to the base plate 1213, a repulsive force of the first magnet on the second magnet in a length direction of the scratching needle 1310 may be maintained at a stable level, so as to ensure a pressure of the scratching needle 1310 on the bottom of the cultivation portion 110 is maintained at a stable level, thereby better improving the uniformity and stability of the scratching.

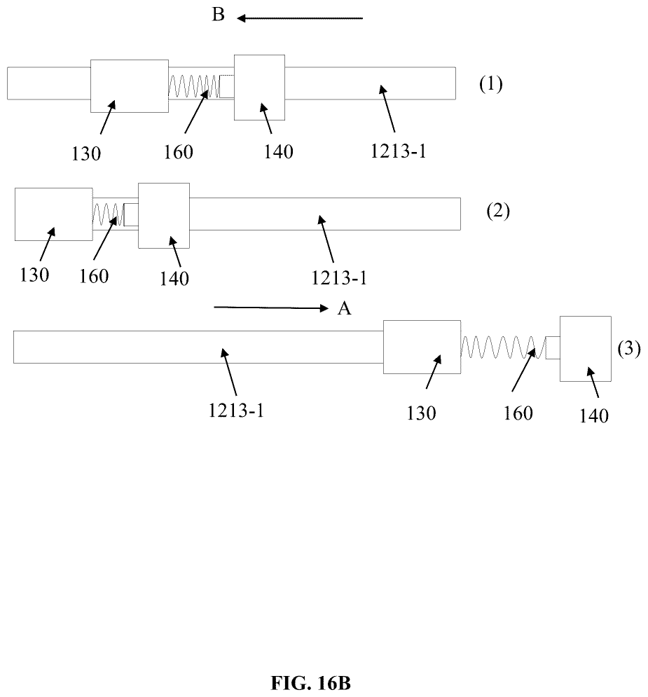

In some embodiments, the scratching portion 120 may further include a driving member 140 and one or more sensors 150. The driving member 140 may be configured to drive the scratching member 130 to scratch along the scratching gap 1213-1, and the one or more sensors may be configured to recognize a pressure that the scratching member 130 is subjected to while scratching and/or a sliding distance of the scratching member 130. Referring to FIG. 16A and FIG. 16B, FIG. 16A and FIG. 16B are schematic diagrams illustrating an exemplary connection structure of the driving member 140 and the scratching member 130 according to some embodiments of the present disclosure.

In some embodiments, the driving member 140 may include a motor. In some embodiments, the sensor 150 may include a pressure sensor, a displacement sensor, etc., or any combination thereof. For example, the pressure sensor may include a piezoresistive pressure sensor, a ceramic pressure sensor, a diffuse silicon pressure sensor, a sapphire pressure sensor, a piezoelectric pressure sensor, or the like. As another example, the displacement sensor may include a strain sensor, an inductive sensor, a differential transformer sensor, eddy current sensing, and a Hall sensor, among others.

In some embodiments, when the sensor 150 is a pressure sensor, it is only necessary to ensure a stable reading of the sensor 150 while scratching, so that the scratching member 130 is subjected to a stable pushing/pulling force, henceforth ensuring uniform scratching. When the sensor 150 is a displacement sensor, it is possible to recognize whether the scratching member 130 has moved a specified distance, or reached an end of a scratch. The end of the scratch refers to the end of the scratching gap 1213-1.

On the other hand, it may be possible to determine whether the scratching has begun by the sensor 150. As shown in FIG. 16A, in some embodiments, the scratching member 130 may be coupled to the driving member 140 via a spring 160, and the spring 160 may be set along the scratching gap 1213-1 (i.e., along AB in FIG. 16A). As shown in FIG. 16B, before starting the scratching, if the scratching member 130 is located at a position shown in FIG. 16B(1), a horizontal position of a tip of the scratching member 130 may be set at a position in which cells in the cultivation portion 110 are not contacted. Then the scratching member 130 is moved to an end of the cultivation portion 110 (e.g., a direction of an arrow pointing to B), if a tension (or pressure) of the spring 160 starts to increase, it means that the scratching member 130 reaches one of ends of the scratching gap 1213-1 (as shown in FIG. 16B (2)), and the horizontal position of the tip of the scratching member 130 may be shifted downward to an optimum scratching height. Then a scratching operation on the cells in the cultivation portion 110 may start, and the scratching operation ends until the pressure (or the tension) of the spring 160 begins to increase (as shown in FIG. 16B (3)). In particular, if a plurality of scratching members 130 are driven at the same time to individually scratch, it is necessary to move down the scratching members 130 to start scratching or to end the scratching only when all tensions or pressures of the springs 160 start to increase. In some embodiments, the driving member 140 may be directly coupled to the scratching member 130, i.e., there is no spring 160.

In other embodiments, the sensor 150 may be disposed along a length direction of the scratching member 130. Thus, the sensor 150 may recognize the pressure that the scratching member 130 is subjected to when the scratching member scratches. Ensuring that the sensor 150 has a stable reading when scratching ensures that the scratching member 130 is under a stable amount of pressure, which ensures that the scratching is even.

In some embodiments, the cell scratching device 101 may include the cultivation portion 110, the scratching member 130, a processor, a scratching table, and a motor. The cultivation portion 110 may include a petri dish. The motor may be configured to drive the scratching member 130 to work, and the processor may be configured to control the motor to work. In some embodiments, the scratching table may include a sample table, a petri dish fixation device, and a petri dish identification device. The sample table may be configured to provide an operational support platform for cell scratching, and the petri dish fixation device and the petri dish identification device are mounted to the sample table. The petri dish fixation device may be configured to fix a cell cultivation dish, and the petri dish identification device may be configured to identify information such as a position and/or model number of the petri dish.

In some embodiments, the petri dish fixation device may include a groove that matches the petri dish or a support fixation frame. In some embodiments, the petri dish identification device may comprise a sensor (e.g., a pressure sensor, etc.) disposed on the sample stage, and/or a camera device (i.e., recognizing the position of the petri dish by image).

While performing a scratching operation, the petri dish may be fixed to a designated position on the sample stage by the petri dish fixation device. When fixing, the petri dish may be manually placed at the designated position. In other embodiments, the scratching table may further include a petri dish moving mechanism, the petri dish fixation device being mounted to the petri dish moving mechanism. The processor may recognize the position of the petri dish by the petri dish identification device to control the petri dish moving mechanism to move the petri dish to the designated position on the sample table. For example, the petri dish moving mechanism may be a grid-like guide rail, and the petri dish fixation device is slidingly connected to the guide rail, and the petri dish fixation device moves in a plane of the sample stage by switching between different guide rails (a sliding structure may be referred to the spherical slider and guide rails matching the spherical slider). The petri dish moving mechanism may also be set up as a two-stage guide rail, with a first-stage guide rail fixed to the sample stage along an X-axis direction, a second-stage guide rail slidingly connected to the first-stage guide rail and the second-stage guide rail arranged along a Y-axis direction, and the petri dish fixing device being slidingly connected to the second-stage guide rail. A position in the X-axis direction is adjusted by the stage-one guide rail, and a position in the Y-axis direction is adjusted by the second-stage guide rail, so as to realize that an arbitrary position in an XY plane is adjustable.

In some embodiments, the petri dish identification device may send a model number of an identified petri dish to the processor, and the processor selects a preset scratching scheme based on the model number of the petri dish. In some embodiments, the scratching scheme may include an interval corresponding to a scratching gap, a count of scratches, or the like. In some embodiments, the scratching scheme may be determined based on an experimental requirement entered by a user (e.g., an experimenter).

In some embodiments, the processor may determine the scratching scheme and a photographing scheme based on a cultivation requirement and a measurement requirement by matching vectors in a vector database.

The cultivation requirement refers to a requirement associated with cell cultivation. In some embodiments, the cultivation requirement may include at least one of a model of a petri dish, a cell type of cultivated cells, a cultivation environment (e.g., a cultivation temperature, a cultivation humidity, etc.).

The measurement requirement refers to a requirement associated with photographing the scratch. In some embodiments, the measurement requirement may include at least one of measurement accuracy, or a measurement data volume. The measurement accuracy is a requirement for an operation accuracy of an image analyzing device (e.g., the cell image analysis device) required by a user. The operation accuracy may be expressed using a pre-divided quality level, such as quality level 1 to 5, and the higher the level, the higher the requirement for an operation accuracy. The measurement data volume is a richness degree of data desired by the user, and the measurement data volume may be expressed using a pre-divided data level, such as data level 1 to 5, and the higher the level, the more measurement data volume.

In some embodiments, the scratching scheme may include an interval corresponding to a scratching gap, a count of scratches, a scratch shape, a scratch speed, etc.

The photographing scheme is a scheme for photographing a scratch. In some embodiments, the photographing scheme may include a photographing parameter, a photographing time point, a photographing path, or the like. The photographing path is a sequence in which at least one view field of at least one scratch is photographed, the photographing path may be composed of a position coordinate of the at least one view field. More description of the photographing parameter and the photographing time point may be found in FIG. 2A and its related description.

Merely by way of example, the vector database is constructed based on a plurality of historical cultivation requirements and historical measurement requirements and corresponding historical scratching schemes and historical photographing schemes. For example, the vector database may include a plurality of reference vectors and reference scratching schemes and reference photographing schemes corresponding to reference vectors. The processor may construct the vector database by a plurality of manners. For example, the processor may construct vectors by manners such as TF-IDF (Term Frequency-Inverse Document Frequency), One-Hot, Word2Vec, etc., and then process and store vector data by manners such as hash indexing, clustering indexing, or the like.

In some embodiments, the processor may construct a target feature vector based on the cultivation requirement and the measurement requirement; based on the target feature vector, the processor may determine a reference vector that satisfies a predetermined retrieval condition as a target vector by the vector database. A reference scratching scheme and a reference photographing scheme corresponding to the target vector are determined as a final scratching scheme and a final photographing scheme. The predetermined retrieval condition is a minimum vector distance to the target feature vector.

In some embodiments, the processor may calculate an initial scratching position and an end scratching position for each scratch according to a corresponding scratching scheme, and control the motor to drive the scratching member 130 to move according to the initial scratching position and the end scratching position to performing a scratching operation. In some embodiments, whether a current scratching is over may be recognized by providing a transverse pressure sensor on a scratching device. For example, when the pressure sensor senses an increase in a transverse pressure when the scratching member 130 scratches an edge of the petri dish, which represents the end of the current scratching, and the processor may control the scratching member 130 to move to an initial scratching position of a next scratch for a next scratching operation.

In other embodiments, the sample stage may also be driven by another motor to move back and forth in a plane along an XY axis. For example, a bottom of the sample stage may also be a two-stage guide rail setup (please refer to the above descriptions). The processor may control the motor to drive the sample stage to move in the XY plane to perform scratching operation.

In other embodiments, the processor may also control the petri dish moving mechanism to move the petri dish in the XY plane to enable the scratching operation.

In some embodiments, the cell scratching device 101 may be made of glass, metal, plastic, resin, or the like. The cell scratching device 101 of the present disclosure is mainly applied to a cell scratching experiment, a transparent material is convenient for observing the scratching effect, and at the same time, utensils of the cell scratching experiment need to be autoclaved frequently, the material needs to be resistant to high temperature and high pressure, therefore a scratching cover plate is preferably a glass product.

Possible beneficial effects of the cell scratching device disclosed in the present disclosure include, but are not limited to, (1) the scratching cover plate and the cultivation portion are mounted by the position-limiting structure, which reduces the lateral swing of the scratching portion during the scratching operation, thereby makings a shape of the scratch more regular; (2) the scratching member moves along the scratching gap, so that the formed scratch is straight and intervals between different scratches are uniform, which is convenient for subsequent observation; (3) the scratching member cooperates with the scratching cover plate through position-limiting, which restricts the movement of the scratching member in a vertical direction, so that a force of the scratching member acting on the cells is just moderate, which prevents the scratch from being incomplete due to too little force and not scratches the cultivation portion due to too much force, so as to ensure the stability of the scratch; (4) the scratching member is driven by the motor, and the sensor detects the pressure and displacement of the scratching member, henceforth realizing an automatic scratching, reducing labor cost, and improving efficiency, and at the same time ensuring the uniformity and stability of the scratch.

Possible beneficial effects of the system for determining a cell migration capacity disclosed in the present disclosure include, but are not limited to, (1) the cell image analysis device is applied to automatically photograph and analyze a scratch, which is simple and convenient to operate, thereby reducing labor cost and improving the efficiency of photographing and analyzing; (2) by combining the use of the cell scratching device and the cell image analysis device, when analyzing a migration situation of the cells of the scratch, it is possible to accurately locate the scratch, which enables fixed-point continuous photography, imaging, and analysis, ensuring the scientificity and credibility of the experimental results; (3) by controlling the movement directions of the sample stage or the photographing module, the cumulative observation and analysis of a single scratch and the comparative observation and analysis of multiple scratches can be realized. It should be noted that different embodiments may have different beneficial effects, and in different embodiments, the possible beneficial effects may be any one or a combination of the above, or any other possible beneficial effect.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method for determining a cell migration capacity, comprising:
performing a scratching operation on cells through a cell scratching device to form at least one scratch;
positioning and photographing the at least one scratch by a cell image analysis device to obtain at least one image of the at least one scratch; and
determining a migration capacity of the cells based on the at least one image of the at least one scratch.

2. (canceled)

3. The method of claim 1, wherein
a fluorescent marker is provided at an endpoint of the at least one scratch, and the positioning and photographing the at least one scratch by the cell image analysis device includes: determining, by the cell image analysis device, the endpoint of the at least one scratch by tracing the fluorescent marker to achieve the positioning and photographing of the at least one scratch, or
a bottom of the at least one scratch is provided with an irradiated light, and the positioning and photographing the at least one scratch by the cell image analysis device includes: positioning, by the cell image analysis device, the at least one scratch based on a light transmission performance of the at least one scratch to achieve the positioning and photographing the at least one scratch.

4. (canceled)

5. The method of claim 1, wherein the cell image analysis device includes at least one local camera and at least one global camera, and the positioning and photographing the at least one scratch by the cell image analysis device includes:
photographing a global image of the at least one scratch by the global camera;
determining scratch information of the at least one scratch based on the global image; and
photographing, by the local camera, images of a plurality of view fields of the at least one scratch based on the scratch information to achieve the positioning and photographing of the at least one scratch.

6. The method of claim 1, wherein the cell image analysis device performs the positioning and photographing of the at least one scratch based on a same photographing parameter to obtain the at least one image of the at least one scratch, the photographing parameter including at least one of a sample feed coordinate, a total photographing length of the at least one scratch, a count of view fields of the at least one scratch, an exposure degree, or a photographing focal length.

7-10. (canceled)

11. The method of claim 1, wherein the cell scratching device includes a cultivation portion and a scratching portion, the cultivation portion is configured to cultivate the cells, the scratching portion includes a scratching cover plate and a scratching member, the scratching cover plate includes a base plate, a connecting member, and a position-limiting structure that are connected sequentially, the base plate and the position-limiting structure are respectively located at two ends of the connecting member, and the base plate is provided with at least one scratching gap.

12. The method of claim 11, wherein the performing a scratching operation on the cells through a cell scratching device to form at least one scratch includes:
securing the scratching cover plate to the cultivation portion through the position-limiting structure; and
performing the scratching operation on the cells by inserting the scratching member into the at least one scratching gap and moving the scratching member along one end of the at least one scratching gap to the other end of the at least one scratching gap, wherein a position of the at least one scratch is determined by a position of the at least one scratching gap.

13. The method of claim 12, wherein method further includes:
automatically photographing, through the cell image analysis device, the at least one scratch based on the photographing parameter and the position of the at least one scratch to obtain a plurality of images of the at least one scratch, wherein each image of the plurality of images corresponds to a preset time point; and
determining the migration capacity of the cells based on the plurality of images of the at least one scratch and a plurality of preset time points corresponding to the plurality of images.

14. The method of claim 13, wherein a plurality of scratches is formed after the scratching operation is performed on the cells, the plurality of scratches includes a first scratch and a second scratch, and the automatically photographing, through the cell image analysis device, the at least one scratch based on the photographing parameter and the position of the at least one scratch includes:
controlling the cell image analysis device to automatically photograph the first scratch based on the photographing parameter and a position of the first scratch;
determining a positional relationship between the first scratch and the second scratch based on a positional relationship between a first scratching gap corresponding to the first scratch and a second scratching gap corresponding to the second scratch; and
controlling the cell image analysis device to automatically photograph the second scratch based on the positional relationship between the first scratch and the second scratch and the photographing parameter.

15. The method of claim 13, wherein the cell image analysis device includes a sample stage and a photographing module, and the automatically photographing, through the cell image analysis device, the at least one scratch based on the photographing parameter and the position of the at least one scratch to obtain a plurality of images of the at least one scratch includes:
at each of the plurality of preset time points,
positioning the cultivation portion of the cell scratching device at a target position of the sample stage; and
controlling the photographing module to automatically photograph the at least one scratch based on the photographing parameter and the position of the at least one scratch to obtain at least one image of the at least one scratch at the preset time point.

16-18. (canceled)

19. The method of claim 15, wherein the sample stage is provided with a moving mechanism, and the positioning the cultivation portion of the cell scratching device at the target position of the sample stage includes:
obtaining an image of the cultivation portion by photographing the cultivation portion through the cell image analysis device;
determining a center point of the cultivation portion by analyzing the image of the cultivation portion;
determining a movement direction and a movement distance of the cultivation portion based on a positional relationship between the center point of the cultivation portion and a zero position of the sample stage; and
positioning, through the moving mechanism, the cultivation portion at the target position of the sample stage based on the movement direction and the movement distance of the cultivation portion.

20. The method of claim 15, wherein a bottom of the cultivation portion is provided with a first adapter for scratch positioning, a bottom of the sample stage is provided with a corresponding second adapter, and the controlling the photographing module to automatically photograph the at least one scratch based on the photographing parameter and the position of the at least one scratch includes:
automatically photograph the at least one scratch by matching the first adapter with the second adapter.

21. The method of claim 15, wherein
the controlling the photographing module to automatically photograph the at least one scratch based on the photographing parameter and the position of the at least one scratch to obtain the at least one image of the at least one scratch at the preset time point includes: controlling the sample stage or the photographing module to move along a direction parallel to an extending direction of the at least one scratch to obtain a plurality of images of multiple fields of view of the at least one scratch along the extension direction, or
a plurality of scratches are formed after the scratching operation is performed on the cells, the plurality of scratches extending in a direction parallel to each other, and the controlling the photographing module to automatically photograph the at least one scratch based on the photographing parameter and the position of the at least one scratch to obtain at least one image of the at least one scratch at the preset time point includes: controlling the sample stage or the photographing module to move along a direction perpendicular to an extending direction of the plurality of scratches to obtain a plurality of images of the plurality of scratches.

22. (canceled)

23. The method of claim 13, wherein the determining the migration capacity of the cells based on the plurality of images of the at least one scratch and a plurality of preset time points corresponding to the plurality of images includes:
determining a migration distance or a migration area of the cells based on the plurality of images; and
determining the migration capacity of the cells based on the migration distance or the migration area, and the plurality of preset time points.

24-25. (canceled)

26. The method of claim 13, wherein the at least one scratch includes a plurality of scratches, and the determining the migration capacity of the cells based on the plurality of images of the at least one scratch and a plurality of preset time points corresponding to the plurality of images includes:
determining migration abilities of a plurality of candidate cells corresponding to the plurality of scratches; and
determining the migration capacity of the cells based on an average value of the migration abilities of the plurality of candidate cells.

27. The method of claim 1, wherein the positioning and photographing the at least one scratch by the cell image analysis device to obtain at least one image of the at least one scratch includes:
inputting a position coordinate of a first photographing view field of the at least one scratch into the cell image analysis device;
determining, by the cell image analysis device, a sample feed coordinate of a sample stage based on a coordinate of a zero position of the sample stage and the position coordinate of the first photographing view field; and
controlling, by the cell image analysis device, a movement direction and/or a movement distance of the sample stage based on the sample feed coordinate of the sample stage to photograph, by the cell image analysis device, the first photographing view field of the at least one scratch on the sample stage.

28. The method of claim 1, wherein the positioning and photographing the at least one scratch by the cell image analysis device to obtain at least one image of the at least one scratch includes:
obtaining a total photographing length and a count of view fields of a first scratch to be photographed of the at least one scratch;
determining a total photographing length and a count of view fields of the other scratches of the at least one scratch based on a positional relationship between the other scratches and the first scratch to be photographed; and
positioning and photographing the at least one scratch sequentially based on a total photographing length and a count of view fields of the at least one scratch.

29. The method of claim 1, wherein the positioning and photographing the at least one scratch by the cell image analysis device to obtain at least one image of the at least one scratch includes:
inputting position coordinates of a plurality of view fields of the at least one scratch into the cell image analysis device; and
automatically photographing, by the cell image analysis device, the plurality of view fields based on the position coordinates of the plurality of view fields of the at least one scratch.

30-31. (canceled)

32. A system for determining a migration cell capacity, comprising:
a cell scratching device configured to perform a scratching operation on cells to form at least one scratch; and
a cell image analysis device configured to:
position and photograph the at least one scratch to obtain at least one image of the at least one scratch; and
determine a migration capacity of the cells based on the at least one image of the at least one scratch to determine a migration capacity of the cells.

33-36. (canceled)

37. The system of claim 32, wherein the cell scratching device includes:
a scratching portion, the scratching portion includes a scratching cover plate, a scratching member, a driving member, and at least one sensor, the scratching cover plate includes a base plate, a connecting member, and a position-limiting structure that are connected sequentially, the base plate is provided with at least one scratching gap, the driving member is configured to drive the scratching member to move along the at least one scratching gap, the at least one sensor is configured to identify a pressure subjected by the scratching member during the scratching operation and/or a sliding distance of the scratching member.

38-54. (canceled)

55. A control system for determining a cell migration capacity, comprising:
at least one storage device configured to store a set of instructions; and
at least one processor being in communication with the at least one storage device, and when executing the stored instructions, the at least one processor causes the system to:
control a cell scratching device to perform a scratching operation on cells to form at least one scratch; and
position and photograph the at least one scratch by a cell image analysis device to obtain at least one image of the at least one scratch; and
determining a migration capacity of the cells based on the at least one image of the at least one scratch.

56. (canceled)
